(19) United States
(12) Patent Application Publication
(10) Pub. No.: US 2009/0036384 A1
(43) Pub. Date: Feb. 5, 2009

(54) INCREASING PREGNANCY RATES

(75) Inventors: Alan Richard Bell, Woollahra (AU); Richard W. Shephard, Maffra (AU); Salvatore Iemma, Seaforth (AU); Vladimir Stajic, Kirrawee (AU)

Correspondence Address: WOOD, HERRON & EVANS, LLP, 2700 CAREW TOWER, 441 VINE STREET, CINCINNATI, OH, 45202, US

(73) Assignee: PARNELL LABORATORIES (AUST) PTY LIMITED, Alexandria, New South Wales (AU)

(21) Appl. No.: 12/095,456

(22) PCT Filed: Dec. 1, 2006
(86) PCT No.: PCT/AU2006/001832

§ 371 Date: Oct. 21, 2008

(30) Foreign Application Priority Data
Dec. 2, 2005 (AU) 2005906775

(51) Int. Cl. A61K 38/09

(52) U.S. Cl. 514/15

(57) ABSTRACT

The invention relates to a stable formulation for controlling oestrus in a subject. The formulation comprises Gonadotrophin Releasing Hormone or an acceptable salt or analogue thereof, progesterone or an analogue thereof, and an acceptable carrier.

### TECHNICAL FIELD

The present invention relates to a formulation for use in increasing the rate of pregnancy in an animal.

### BACKGROUND OF THE INVENTION

Throughout the dairy industry worldwide, control of the ovulation cycle in cows is essential to ensure a regular supply of lactating cows. Reproductive performance remains an area of dairy management that must be optimised to ensure continued productivity and a sustainable operation. A cow must be pregnant by 85 days postpartum in order to achieve optimum milk production and calve every 12½ months.

Management of reproduction is achieved by treatment of the cows with a variety of hormone interventions at particular times followed by artificial insemination. There are several hormone intervention systems in use throughout the world. Despite the extensive research into these procedures, a disadvantage with them remains that only approximately 50% of the cows receiving these treatments become pregnant. The reasons for this include the fact that the patterns and timing of ovulation in cows differs so that not all cows have mature ova at the time of artificial insemination. There is a great deal of ongoing research to try to increase the number of cows that do become pregnant following hormone intervention programmes.

The oestrus cycle of the cow is divided into four stages. These are:
Metoestrus—this is from the cessation of oestrus (standing heat) until a functional corpus luteum (CL) has formed after ovulation. Metestrus typically lasts 3-5 days
Dioestrus—the period of the functional corpus luteum (CL) and this stage typically lasts 10-14 days
Proestrus—the time of rapid CL regression. This stage typically lasts for 2 to 4 days
Oestrus—the period of sexual receptivity by the female. Oestrus lasts for between 6-24 hours, but is usually of a shorter duration for heifers than for cows.

The development and regression of structures on the ovary is a dynamic process. The process is timed and controlled by a series of cascading and interrelated feedback mechanisms. These feedback systems are complex because the ovaries contain a limited (finite) number of follicles. Therefore, the control system must conserve follicular resources but maximise the number of opportunities for a successful conception and pregnancy during the reproductive life of the animal. These objectives are contradictory therefore the control system must receive input from physiological states such as pregnancy, lactation and nutritional plane and from existing ovarian structures such as corpora lutea and follicles. The system must ensure that a mature follicle is provided for ovulation at a regular interval and that it is timed to ovulate into a receptive and non-pregnant tract.

The process is best understood by first examining the factors that directly exert control on the development and regression of follicles and corpora lutea. When these basic structural cycles are understood, the relationship between the follicular cycle and the life of the corpus luteum becomes apparent from examination of hormones that exert influence upon both structures. Finally, the overall regulation of events and timing within the system can be visualised from an examination of the interplay between the various hormones of reproduction.

The majority of follicles within the ovaries are small, undeveloped and inactive. A follicle must experience significant growth (of around 8-10 days duration) before it can provide an ovum capable of in vivo fertilisation. Once a follicle is recruited for maturation it will either continue development to become capable of ovulation or it will regress and die. Thus the ovary must regularly recruit only a fraction of the total number of follicles for development. Follicle Stimulating Hormone (FSH) initiates the recruitment process. FSH is released from the pituitary gland under the influence of Gonadotrophin Releasing Hormone (GnRH) which is released from the hypothalamus. This cascade recruits a small number of follicles to begin development at regular intervals.

The ongoing growth of the recruited follicles is hormone dependent. In the early stages of growth, ongoing FSH release provides this tropic effect on the follicles. However, the FSH stimulus gradually declines over the next 4-5 days due to negative feedback provided by the developing follicles. The growing follicles produce increasing amounts of oestradiol and inhibin and these hormones feedback onto the hypothalamus-pituitary junction resulting in decreased release of GnRH and FSH. In the absence of any further stimulus, follicle development is halted at around 4-5 days and most of the follicles begin to die (become atretic).

The largest follicle of the recruited crop will continue to develop whilst the smaller follicles become atretic. This occurs because the dominant follicle develops luteinising hormone (LH) receptors and LH has a tropic effect. LH is a pituitary hormone whose release is also controlled by GnRH. GnRH release is itself subject to negative feedback by ovarian hormones, with progesterone providing the strongest inhibition to further GnRH and therefore LH release. The major natural source of progesterone is from an active corpus luteum (CL). An active CL is present during pregnancy and during the luteal phase of the ovarian cycle.

The point in development of the follicular wave when the largest follicle develops capacity to be stimulated to grow by LH is called deviation. Deviation typically occurs around 4-5 days into the follicular wave, when the largest follicle is around 8 mm in diameter and it occurs rapidly—within an 8-hour period. The mechanism of deviation appears to be the development of LH sensitivity within the dominant follicle. Deviation tends to occur when there is a nadir in FSH levels (arising from the negative feedback effect of oestradiol and inhibin on FSH release). Thus further follicle development is wholly dependant upon LH: the largest follicle continues to grow and the other smaller follicles begin to regress.

The developing dominant follicle increases the production of oestradiol and inhibin and these hormones contribute to a reduction in LH release via their negative feedback on GnRH release. Thus, the rate of follicular growth reduces as the dominant follicle approaches ovulatory size. If no further LH stimulus is received, this follicle will also undergo atresia, typically around day 7-9 of the follicle wave. However, if circulating progesterone levels are low (e.g. at proestrus), there is a temporary reversal of the negative oestradiol feedback loop and a large surge of LH is released from the pituitary. This surge of LH release promotes final development of the follicle. Growth continues to between 10-20 mm in diameter: the typical follicle diameter at ovulation is 15 mm for heifers and 17.5 mm for cows, and ovulation is initiated to occur at around 24 hours later. The ovulatory follicle then further increases the production and release of oestradiol and this initiates the onset of oestrus.

When a follicular wave regresses, circulating levels of oestradiol and inhibin are also reduced. This allows for release of FSH to occur again, thus initiating recruitment of another batch of follicles. This sequential process of follicle recruitment, growth for 4-5 days, atresia of most of the follicles but recruitment of a single dominant follicle with growth for another 3-4 days is called a follicular wave. Follicular waves occur in a sequential manner, one after the other and are not strongly influenced by pregnancy, lactation, age or physiological status. The follicular wave is the metronome of the ovarian cycle. Hormone intervention systems that control (reset) follicular waves are commonly characterised by accurate timing of subsequent ovulation, heat and ovarian events.

The luteal phase is characterised by a period of progesterone dominance. A corpus luteum (CL) forms from the remnants of the ovulated follicle. The developing CL grows rapidly, increasing the production of progesterone until it is mature at around 4-5 days after ovulation. The mature CL remains viable and continues to produce progesterone for another 10-12 days. Beyond this point, the CL itself requires further tropism to retain functionality and to prevent regression. This must be provided by a pregnancy stimulus. In the absence of pregnancy, the combined effect of the lack of ongoing tropism plus the release of prostaglandin by the endometrium results in rapid destruction of the CL and cessation of progesterone production.

The precipitous drop in progesterone levels that occur when the CL regresses, allows GnRH and LH levels to rise again. Thus, the level of progesterone that is present at the end of the follicular wave determines the fate of the dominant follicle of that wave. If progesterone levels are high (e.g. during pregnancy or the luteal phase), then the dominant follicle will regress due to an absence of LH stimulus. If progesterone levels are low, the dominant follicle will continue to grow under the stimulation of LH leading to ovulation within a few days. There is no fixed timing between the end of the luteal phase and the stage of the follicular wave. This is of no significance in natural mating systems as the dominant follicle will experience unimpeded growth until it is mature and capable of ovulation in the absence of progesterone. It is important for hormonal intervention programmes that exert control over the cycle via progesterone (i.e. by controlling the luteal phase). Systems based on progesterone alone provide a wide window of time for ovulation of the dominant follicle.

Therefore, the stage of the luteal cycle determines if an ovulation will occur at the end of the follicle wave, but the stage of the follicular cycle determines the timing of ovulation.

Cows typically either have 2 or 3 follicular waves per cycle. Cows with 2-wave cycles typically have a follicular wave every 9 days resulting in an oestrous cycle of 18 days. Cows with a 3-wave cycle have a follicular wave every 8 days resulting in an oestrous cycle of 24 days. The sequence of events for a 2-wave cow is presented in FIG. 1.

Synchrony-based reproductive programmes provide an opportunity to increase the rate of matings within a herd resulting in more pregnancies early in the mating period and often provide an increase in the overall pregnancy rate. The successful implementation of a synchrony programme within a herd is challenging. It requires careful planning, with consideration made for: the logistics of mating large number of cows over a short period of time, synchronised returns when changing to the natural mating system, calving period planning and the increased demand for skilled labour during critical periods.

The traditional synchrony programme employed by seasonal dairy farmers used prostaglandin. The double prostaglandin (DPG) injection system, consisting of two injections of prostaglandin administered 11-14 days apart, is designed to synchronise ovulation in the majority of herd members through early regression of the CL (corpus luteum). Prostaglandin systems are designed for cycling cows; there is no effect on anovulatory anoestrous cows. Prostaglandin is not effective during the period of the cycle when there is no active CL and it is not effective at destroying a forming CL (the first 5 days after ovulation). Thus, prostaglandin is effective for only around ⅔rds of the cycle. The differences in cycle length and follicular wave length between 2-wave and 3-wave cows result in variation in susceptibility to both the first and second PG injection within this programme. Stochastic modelling of this natural variation indicates that only around 70-80% of cycling cows respond to the DPG protocol by ovulating in the five day period immediately after the second PG injection. Cows that respond to the DPG programme must be detected in oestrus—wastage in this area can reduce the proportion of cycling cows that are successfully inseminated after a DPG programme to between 60-70%. Therefore it seems that DPG synchrony programmes are less effective than originally envisaged in cycling cows.

It appears that distribution of 2-wave and 3-wave follicular cycles are changing within the population. Around 40% of cows have a cycle length less than 21-days in duration (see FIG. 2). It is also not known if cows can change between 2- or 3-wave cycles over time. High milk producing cows are more likely to be 2-wave cows and it is thought that 2-wave cows may be less fertile than 3-wave cows. A commercial hormone intervention programme would preferably be able to synchronise both 2-wave and 3-wave cows, which is a major challenge. Failure to do so may contribute to the low success rate of many of those programmes.

One possible reason for reduced fertility in 2-wave cows is that the dominant follicle that forms in the second wave in the ovarian cycle is often smaller than those formed from the first wave or the third wave (for 3-wave cows). The second wave develops during the height of the luteal phase and therefore develops during a period of progesterone dominance. This progesterone dominance results in a reduced magnitude and amplitude of LH release thereby resulting in a smaller dominant follicle (see FIG. 1). The dominant follicle of the third wave of the cycle undergoes much of its growth and development during the regression of the CL. This is a period of reduced progesterone and therefore increased LH levels and this stimulates the development of a larger ovulatory follicle. This variability places further pressures on any hormone intervention programme.

A further factor leading to the reduced efficacy of hormone intervention programmes is the differences in cycle lengths in individual cows as illustrated in FIG. 2. The differences in cycle lengths make the timing of the artificial insemination extremely difficult in order to fertilise all cows.

Oestrus is initiated by the combined effect of the rapid fall in progesterone levels from the regressing CL and the rapid increase in estradiol levels that follow the LH surge. Ovulation is initiated by the LH surge and typically occurs around 28 hours after the surge which is generally around 24 hours after the start of standing oestrus. Oestrus duration is an average of around 8 hours for cows and 5 hours for heifers. This has reduced from an average duration of 12 hours in cows recorded ten years ago. The reduced oestrus display has implications for systems of mating that rely on heat detection. Two periods of heat detection that are evenly (12-hourly) spaced throughout the day would reliably detect all cows on heat when the oestrus duration is 12 hours. However this increases to a requirement for three periods of heat detection evenly spaced (8-hourly) through the day in order to detect all cows that have an average oestrus duration of 8 hours. Up to five detection periods may be needed to detect all heifers. It is difficult to heat detect cows at pasture at night, therefore the reduction of the average oestrus duration to less than the period of darkness (12 hours) presents a challenge to artificial mating systems that rely on heat detection within pasture based systems.

It is clear that there are a large number of variable factors that have to be considered when developing a hormone intervention programme that leads to a large proportion of the cows becoming pregnant following fixed time artificial insemination.

### OBJECT OF THE INVENTION

It is an object of the present invention to overcome or substantially ameliorate at least one of the above disadvantages.

### SUMMARY OF THE INVENTION

In a first aspect of the invention there is provided a stable formulation for controlling oestrus in a subject comprising:
GnRH (Gonadotrophin Releasing Hormone) or an acceptable salt or analogue thereof;
progesterone or an analogue thereof; and
an acceptable carrier.

In an aspect of the invention there is provided a stable formulation for controlling oestrus in a subject consisting essentially of or consisting of:
GnRH (Gonadotrophin Releasing Hormone) or an acceptable salt or analogue thereof;
progesterone or an analogue thereof; and
an acceptable carrier.

In an aspect of the invention there is provided a stable pharmaceutically acceptable or veterinarily acceptable formulation for controlling oestrus in a subject comprising, consisting essentially of or consisting of:
GnRH (Gonadotrophin Releasing Hormone) or an acceptable salt or analogue thereof;
progesterone or an analogue thereof; and
a pharmaceutically acceptable or veterinarily acceptable carrier.

The formulation may be a solution, a micellar solution, a microemulsion, a miniemulsion or a small particle size emulsion. It may have a droplet size of less than about 0.2 microns, or it may have no droplets (i.e. may be a solution). It may be optically clear, or may be hazy or translucent. It may be not opaque. It may be colourless or slightly yellow or yellow or some other colour. The progesterone or analogue thereof and the GnRH or acceptable salt or analogue thereof may be in solution in the formulation. The formulation may be sterilisable, e.g. terminally sterilisable. It may be sterilizable by means of filtration through a sterilising filter, for example through a filter with pore size of 0.2 microns or less, by UV irradiation or by some other suitable non-thermal sterilisation method. The formulation may or may not comprise a preservative, e.g. an antimicrobial or antibacterial compound. The formulation may be sterile.

The progesterone or analogue may be present in the formulation between about 100 and about 150 mg/ml, or between about 25 and about 100 mg/ml or between about 25 and 150 mg/ml. It may be acceptable for injection. It may be naturally derived or may be synthetically produced. It may comprise BP (British Pharmacopoeia) or USP (United States Pharmacopeia) grade progesterone. The GnRH or salt or analogue thereof may be present in the formulation between about 0.02 and about 0.1 mg/ml. It may be naturally derived or may be synthetically produced. It may be mammalian GnRH or an acceptable salt or analogue thereof. It may be a mammalian GnRH analogue. The GnRH analogue may be in the form of a hormone analogue or salt thereof. It may be buserelin, deslorelin, avorelin, leuprolide, natural LHRH, triptorelin, nafarelin, goselerin or fertirelin or an acceptable salt (e.g. an acetate or a hydrochloride) thereof or a mixture of any two or more of the foregoing, or may be some other suitable analogue. It may be for example buserelin acetate. It may be BP or USP. If the GnRH or progesterone is synthetically produced, it may be structurally identical to the natural hormone.

The formulation may additionally comprise water, or the carrier may comprise water. The water may be acceptable for injection. It may be BP (British Pharmacopoeia) or USP (United States Pharmacopeia) grade water, or may be water for injections or saline, for example. The water may be present in the formulation or in the carrier between about 2 and about 50 mg/ml. The water in the formulations of the present invention may be substituted by some other suitable pharmaceutically or veterinarily acceptable aqueous liquid.

The carrier may comprise a solvent, and may comprise one or more cosolvents. The solvent may be a solvent for the progesterone or analogue thereof, or for the GnRH or salt or analogue thereof, or for both. The carrier and/or solvent and/or cosolvent(s) may be injectable. Each of these may be pharmaceutically and/or veterinarily acceptable. The carrier may be any suitable acceptable carrier capable of forming a sterilisable formulation with the GnRH or salt or analogue thereof and the progesterone or analogue thereof. It may comprise an organic solvent, and may comprise a polar organic solvent. It may comprise a dipolar aprotic solvent such as N-methyl-2-pyrrolidone (NMP), glycerol formal (1,3-dioxan-5-ol and/or 4-(hydroxymethyl)-1,3-dioxolane) or dimethylsulfoxide (DMSO) or a mixture of any two or more of these. The solvent may be at least partially miscible with water. It may be capable of dissolving, or mixing with, at least about 5% (or at least about 4 or 3%) of its weight of water, or it may have said concentration of water therein, e.g. dissolved therein. The carrier may comprise water. It may be a mixture of a solvent and water. It may, for example, comprise the solvent containing between about 2 and about 50 mg water per ml solvent, where the solvent is NMP, glycerol formal or a mixture of these.

The formulation may additionally comprise an injection aid. The injection aid may facilitate injection of the formulation (e.g. the GnRH or salt thereof, or the progesterone or analogue thereof) into an animal. The injection aid may comprise an acceptable (e.g. pharmaceutically, veterinarily or clinically acceptable) surfactant. The injection aid may comprise a polymer. It may comprise a hydrophilic polymer. It may be soluble in the carrier and/or in the solvent. It may be pharmaceutically and/or veterinarily and/or clinically acceptable. It may for example comprise polyvinylpyrrolidone (PVP; Plasdone C15; Povidone). Other examples include polyethylene oxide (PEO) and carboxypolymethylene polymers (e.g. carbomer, carbopol, carboxyvinyl polymers). The injection aid may be present in the formulation between about 10 and about 50 mg/ml. The formulation may comprise a preservative, for example an anti-microbial, antibiotic or antioxidant, and may comprise more than one preservative. The preservative may be pharmaceutically and/or veterinarily acceptable. Alternatively it may have no preservative. The preservative may be soluble in the solvent and/or in the carrier. Suitable preservatives include benzyl alcohol, methyl and propyl hydroxybenzoate, chlorocresol, benzalkonium chloride and benzethonium chloride. Suitable antioxidants include butylated hydroxytoluene (BHT), butylated hydroxyanixole (BHA) propyl gallate (PGal), mono and bi-sulfites, ascorbates, cysteine, sulfoxalates, gentistic acid, glutamates, thyoglycolate and thioglycerol and may comprise a combination of two or more antioxidants. The antioxidant should be an acceptable (e.g. pharmaceutically, veterinarily or clinically acceptable) antioxidant. The preservative, e.g. antioxidant, may be present in a concentration effective for preventing microbial growth in the formulation or to prevent deterioration of the formulation. It may be present at between about 0 and 50 mg/ml, for example about 10 mg/ml. The concentration of the preservative may depend on the nature of the preservative. The formulation may be suitable for use in controlling oestrus in an animal. The subject, i.e. the animal, may be a vertebrate, and may be a domestic vertebrate or a farm vertebrate. The animal may be a ruminant, for example a cow or a heifer.

The formulation may comprise a pH adjusting agent. The pH adjusting agent may comprise an acid or a buffer. The acid may be a monobasic acid, or may be a dibasic acid or a tribasic acid. It may for example comprise phosphoric acid, hydrochloric acid or formic acid. The pH adjusting agent may be sufficient to adjust the pH of the formulation to a pH of between about 4 and about 6 or between about 4 and 5, 5 and 6 or 4.5 and 5.5, e.g. about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9 or 6.

In an embodiment there is provided a solution for controlling oestrus in a subject comprising:
GnRH (Gonadotrophin Releasing Hormone) or an acceptable salt or analogue thereof;
progesterone or an analogue thereof; and
an acceptable carrier.

In another embodiment there is provided a microemulsion for controlling oestrus in a subject comprising:
GnRH (Gonadotrophin Releasing Hormone) or an acceptable salt or analogue thereof;
progesterone or an analogue thereof;
an acceptable carrier; and
an injection aid and/or a preservative.

In another embodiment there is provided a stable formulation comprising:
GnRH acetate;
progesterone;
an injection aid;
water; and
an acceptable solvent;
   said formulation being sterilisable by filtration through a sterilising filter.

In another embodiment there is provided a stable formulation comprising:
GnRH acetate;
progesterone;
a preservative;
an injection aid;
water; and
an acceptable solvent;
   said formulation being sterilisable by filtration through a sterilising filter.

In another embodiment there is provided a stable formulation comprising:
GnRH acetate;
progesterone;
polyvinylpyrrolidone;
water; and
NMP;
   said formulation being sterilisable by filtration through a sterilising filter.

In another embodiment there is provided a stable formulation comprising:
GnRH acetate;
progesterone;
benzyl alcohol;
polyvinylpyrrolidone;
water; and
NMP;
   said formulation being sterilisable by filtration through a sterilising filter.

In another embodiment there is provided a stable formulation comprising:
GnRH acetate (about 0.05 mg/ml);
progesterone BP/USP (about 125 mg/ml);
polyvinylpyrrolidone (about 25 mg/ml);
water (about 5 mg/ml); and
NMP to 100%;
   said formulation being sterilisable by means of filtration through a sterilising filter.

In another embodiment there is provided a stable formulation comprising:
GnRH acetate (about 0.05 mg/ml);
progesterone BP/USP (about 125 mg/ml);
benzyl alcohol (about 10 mg/ml);
polyvinylpyrrolidone (about 25 mg/ml);
water (about 5 mg/ml); and
NMP to 100%;
   said formulation being sterilisable by filtration through a sterilising filter.

In another embodiment there is provided a stable formulation comprising:
GnRH acetate;
progesterone;
polyvinylpyrrolidone;
water; and
glycerol formal;
   said formulation being sterilisable by filtration through a sterilising filter.

In another embodiment there is provided a stable formulation comprising:
GnRH acetate;
progesterone;
benzyl alcohol;
polyvinylpyrrolidone;
water; and
glycerol formal;
   said formulation being sterilisable by filtration through a sterilising filter.

In another embodiment there is provided a stable formulation comprising:
GnRH acetate (about 0.05 mg/ml);
progesterone BP/USP (about 125 mg/ml);
polyvinylpyrrolidone (about 25 mg/ml);
water (about 5 mg/ml); and
glycerol formal to 100%;
   said formulation being sterilisable by means of filtration through a sterilising filter.

In another embodiment there is provided a stable formulation comprising:
GnRH acetate (about 0.05 mg/ml);
progesterone BP/USP (about 125 mg/ml);
benzyl alcohol (about 10 mg/ml);
polyvinyl pyrrolidone (about 25 mg/ml);
water (about 5 mg/ml); and
glycerol formal to 100%;
   said formulation being sterilisable by filtration through a sterilising filter.

In another embodiment there is provided a stable formulation comprising:
GnRH acetate (about 0.05 mg/ml);
progesterone BP/USP (about 75 mg/ml);
Butylated Hydroxytoluene (about 0.2 mg/ml);
Butylated Hydroxyanisole (about 0.2 mg/ml);
phosphoric acid 85% (about 10 mg/ml);
water (about 5 mg/ml); and
N-methylpyrrolidine to 100%;
   said formulation being sterilisable by filtration through a sterilising filter.

In an embodiment there is provided a solution for controlling oestrus in a subject consisting essentially of or consisting of:
GnRH (Gonadotrophin Releasing Hormone) or an acceptable salt or analogue thereof;
progesterone or an analogue thereof; and
an acceptable carrier.

In another embodiment there is provided a microemulsion for controlling oestrus in a subject consisting essentially of or consisting of:
GnRH (Gonadotrophin Releasing Hormone) or an acceptable salt or analogue thereof;
progesterone or an analogue thereof;
an acceptable carrier; and
an injection aid and/or a preservative.

In another embodiment there is provided a stable formulation consisting essentially of or consisting of:
GnRH acetate;
progesterone;
an injection aid;
water; and
an acceptable solvent;
   said formulation being sterilisable by filtration through a sterilising filter.

In another embodiment there is provided a stable formulation consisting essentially of or consisting of:
GnRH acetate;
progesterone;
a preservative;
an injection aid;
water; and
an acceptable solvent;
   said formulation being sterilisable by filtration through a sterilising filter.

In another embodiment there is provided a stable formulation consisting essentially of or consisting of:
GnRH acetate;
progesterone;
polyvinylpyrrolidone;
water; and
NMP;
   said formulation being sterilisable by filtration through a sterilising filter.

In another embodiment there is provided a stable formulation consisting essentially of or consisting of:
GnRH acetate;
progesterone;
benzyl alcohol;
polyvinylpyrrolidone;
water; and
NMP;
   said formulation being sterilisable by filtration through a sterilising filter.

In another embodiment there is provided a stable formulation consisting essentially of or consisting of:
GnRH acetate (about 0.05 mg/ml);
progesterone BP/USP (about 125 mg/ml);
polyvinylpyrrolidone (about 25 mg/ml);
water (about 5 mg/ml); and
NMP to 100%;
   said formulation being sterilisable by means of filtration through a sterilising filter.

In another embodiment there is provided a stable formulation consisting essentially of or consisting of:
GnRH acetate (about 0.05 mg/ml);
progesterone BP/USP (about 125 mg/ml);
benzyl alcohol (about 10 mg/ml);
polyvinylpyrrolidone (about 25 mg/ml);
water (about 5 mg/ml); and
NMP to 100%;
   said formulation being sterilisable by filtration through a sterilising filter.

In another embodiment there is provided a stable formulation consisting essentially of or consisting of:
GnRH acetate;
progesterone;
polyvinylpyrrolidone;
water; and
glycerol formal;
   said formulation being sterilisable by filtration through a sterilising filter.

In another embodiment there is provided a stable formulation consisting essentially of or consisting of:
GnRH acetate;
progesterone;
benzyl alcohol;
polyvinylpyrrolidone;
water; and
glycerol formal;
   said formulation being sterilisable by filtration through a sterilising filter.

In another embodiment there is provided a stable formulation consisting essentially of or consisting of:
GnRH acetate (about 0.05 mg/ml);
progesterone BP/USP (about 125 mg/ml);
polyvinylpyrrolidone (about 25 mg/ml);
water (about 5 mg/ml); and
glycerol formal to 100%;
   said formulation being sterilisable by means of filtration through a sterilising filter.

In another embodiment there is provided a stable formulation consisting essentially of or consisting of:
GnRH acetate (about 0.05 mg/ml);
progesterone BP/USP (about 125 mg/ml);
benzyl alcohol (about 10 mg/ml);
polyvinylpyrrolidone (about 25 mg/ml);
water (about 5 mg/ml); and
glycerol formal to 100%;
   said formulation being sterilisable by filtration through a sterilising filter.

In another embodiment there is provided a stable formulation consisting essentially of or consisting of:
GnRH acetate (about 0.05 mg/ml);
progesterone BP/USP (about 75 mg/ml);
Butylated Hydroxytoluene (about 0.2 mg/ml);
Butylated Hydroxyanisole (about 0.2 mg/ml);
phosphoric acid 85% (about 10 mg/ml);
water (about 5 mg/ml); and
N-methylpyrrolidine to 100%;
   said formulation being sterilisable by filtration through a sterilising filter.

In another embodiment there is provided a stable formulation consisting of:
GnRH acetate (about 0.05 mg/ml);
progesterone BP/USP (about 75 mg/ml);
Butylated Hydroxytoluene (about 0.2 mg/ml);
Butylated Hydroxyanisole (about 0.2 mg/ml);
phosphoric acid 85% (about 10 mg/ml);
water (about 5 mg/ml); and
N-methylpyrrolidine to 100%;
   said formulation being sterilisable by filtration through a sterilising filter.

In another embodiment there is provided a stable formulation comprising:

| | | |
|---|---|---|
| Gonadorelin (as Acetate) BP/Ph Eur/NF | 0.050 | mg/ml |
| Progesterone BP/Ph Eur/NF | 75.0 | mg/ml |
| Water for Injections BP/Ph Eur/NF | 30.0 | mg/ml |
| Phosphoric Acid 85% BP/Ph Eur/NF | 10.0 | mg/ml |
| Butylated Hydroxytoluene | 0.2 | mg/ml |
| Butylated Hydroxyanisole | 0.2 | mg/ml |

| | |
|---|---|
| N-Methyl Pyrrolidine | to 100% |

In another embodiment there is provided a stable formulation consisting of:

| | | |
|---|---|---|
| Gonadorelin (as Acetate) BP/Ph Eur/NF | 0.050 | mg/ml |
| Progesterone BP/Ph Eur/NF | 75.0 | mg/ml |
| Water for Injections BP/Ph Eur/NF | 30.0 | mg/ml |
| Phosphoric Acid 85% BP/Ph Eur/NF | 10.0 | mg/ml |
| Butylated Hydroxytoluene | 0.2 | mg/ml |
| Butylated Hydroxyanisole | 0.2 | mg/ml |

| | |
|---|---|
| N-Methyl Pyrrolidine | to 100%. |

In another embodiment there is provided a stable formulation consisting essentially of:

| | | |
|---|---|---|
| Gonadorelin (as Acetate) BP/Ph Eur/NF | 0.050 | mg/ml |
| Progesterone BP/Ph Eur/NF | 75.0 | mg/ml |
| Water for Injections BP/Ph Eur/NF | 30.0 | mg/ml |
| Phosphoric Acid 85% BP/Ph Eur/NF | 10.0 | mg/ml |
| Butylated Hydroxytoluene | 0.2 | mg/ml |
| Butylated Hydroxyanisole | 0.2 | mg/ml |

| | |
|---|---|
| N-Methyl Pyrrolidine | to 100%. |

In a second aspect of the invention there is provided a unit dose for controlling oestrus, said unit dose comprising between about 200 and about 300 mg or between about 150 and about 300 mg of progesterone or an analogue thereof, between about 0.04 and about 0.2 mg GnRH or an acceptable salt or analogue thereof, and an acceptable carrier. In an aspect of the invention there is provided a unit dose for controlling oestrus, said unit dose consisting essentially of or consisting of between about 200 and about 300 mg or between about 150 and about 300 mg of progesterone or an analogue thereof, between about 0.04 and about 0.2 mg GnRH or an acceptable salt or analogue thereof, and an acceptable carrier. In an aspect of the invention there is provided a pharmaceutically acceptable or veterinarily acceptable unit dose for controlling oestrus, said unit dose consisting essentially of or consisting of between about 200 and about 300 mg or between about 150 and about 300 mg of progesterone or an analogue thereof, between about 0.04 and about 0.2 mg GnRH or an acceptable salt or analogue thereof, and a pharmaceutically acceptable or veterinarily acceptable carrier. The progesterone or analogue thereof and the GnRH or acceptable salt or analogue thereof may be in solution in the unit dose. The unit dose may have a volume of between about 1.5 and 4 ml, or may have a volume of greater than 4 ml. The unit dose may comprise between about 1.5 and about 4 ml of the formulation of the first aspect of the invention. The unit dose may comprise between about 1.5 and 3 ml or between about 1.8 and 2.2 ml of the formulation, and may comprise about 2 ml of the formulation. The unit dose may be sterile. The unit dose may be terminally sterilised. It may be filtered through a sterilising filter, such as a 0.2 micron filter or a filter of pore size less than 0.2 microns. The filter may have an absolute pore size cutoff of 0.2 microns or less than 0.2 microns. The unit dose may therefore not contain particles greater than 0.2 microns. The unit dose may be housed within a container, for example a vial, a phial, an ampoule or a syringe, said container having a sufficient volume for the required quantity of the formulation (i.e. at least between about 1.5 and 4 ml). There is therefore also provided a container having a sterile unit dose therein, said unit dose being according to the second aspect. The unit dose may comprise a preservative. The formulation may comprise a preservative, for example an anti-microbial, antibiotic or antioxidant, and may comprise more than one preservative. The preservative may be pharmaceutically and/or veterinarily acceptable. Alternatively it may have no preservative. There may be between about 0 and about 100 mg or more in the unit dose. The unit dose may comprise an injection aid. There may be between about 20 and about 100 mg or more of the injection aid (if present) in the unit dose.

In an embodiment there is provided a unit dose for controlling oestrus, said unit dose comprising progesterone BP/USP (about 200 to about 300 mg, for example about 250 mg), GnRH acetate (about 0.04 to about 0.2 mg, for example about 0.1 mg) and an acceptable carrier. The volume of the unit dose may be about 1.5, 2, 2.5, 3, 3.5 or 4 ml, or some other volume. The progesterone and the GnRH may be in solution in the unit dose.

In another embodiment there is provided a unit dose for controlling oestrus, said unit dose comprising:

| | | |
|---|---|---|
| Progesterone BP/USP | 250 | mg |
| GnRH Acetate | 0.1 | mg |
| Plasdone C15 | 50 | mg |
| Water | 10 | mg |
| N-methyl-2-pyrrolidone | to 2.0 | mL |

In another embodiment there is provided a unit dose for controlling oestrus, said unit dose comprising:

| | | |
|---|---|---|
| Progesterone BP/USP | 250 | mg |
| GnRH Acetate | 0.1 | mg |
| Benzyl Alcohol | 20 | mg |
| Plasdone C15 | 50 | mg |
| Water | 10 | mg |
| N-methyl-2-pyrrolidone | to 2.0 | mL |

In another embodiment there is provided a unit dose for controlling oestrus, said unit dose comprising:

| | | |
|---|---|---|
| Progesterone BP/USP | 250 | mg |
| GnRH Acetate | 0.1 | mg |
| Plasdone C15 | 50 | mg |
| Water | 10 | mg |
| Glycerol formal | to 2.0 | mL |

In another embodiment there is provided a unit dose for controlling oestrus, said unit dose comprising:

| | | |
|---|---|---|
| Progesterone BP/USP | 250 | mg |
| GnRH Acetate | 0.1 | mg |
| Benzyl Alcohol | 20 | mg |
| Plasdone C15 | 50 | mg |
| Water | 10 | mg |
| Glycerol formal | to 2.0 | mL |

In another embodiment there is provided a unit dose for controlling oestrus, said unit dose consisting essentially of or consisting of:

| | | |
|---|---|---|
| Progesterone BP/USP | 250 | mg |
| GnRH Acetate | 0.1 | mg |
| Plasdone C15 | 50 | mg |
| Water | 10 | mg |
| N-methyl-2-pyrrolidone | to 2.0 | mL |

In another embodiment there is provided a unit dose for controlling oestrus, said unit dose consisting essentially of or consisting of:

| | | |
|---|---|---|
| Progesterone BP/USP | 250 | mg |
| GnRH Acetate | 0.1 | mg |
| Benzyl Alcohol | 20 | mg |
| Plasdone C15 | 50 | mg |
| Water | 10 | mg |
| N-methyl-2-pyrrolidone | to 2.0 | mL |

In another embodiment there is provided a unit dose for controlling oestrus, said unit dose consisting essentially of or consisting of:

| | | |
|---|---|---|
| Progesterone BP/USP | 250 | mg |
| GnRH Acetate | 0.1 | mg |
| Plasdone C15 | 50 | mg |
| Water | 10 | mg |
| Glycerol formal | to 2.0 | mL |

In another embodiment there is provided a unit dose for controlling oestrus, said unit dose consisting essentially of or consisting of:

| | | |
|---|---|---|
| Progesterone BP/USP | 250 | mg |
| GnRH Acetate | 0.1 | mg |
| Benzyl Alcohol | 20 | mg |
| Plasdone C15 | 50 | mg |
| Water | 10 | mg |
| Glycerol formal | to 2.0 | mL |

In another embodiment there is provided a unit dose for controlling oestrus, said unit dose comprising:

| | | |
|---|---|---|
| Gonadorelin (as Acetate) BP/Ph Eur/NF | 0.10 | mg |
| Progesterone BP/Ph Eur/NF | 150.0 | mg |
| Water for Injections BP/Ph Eur/NF | 60.0 | mg |
| Phosphoric Acid 85% BP/Ph Eur/NF | 20.0 | mg |
| Butylated Hydroxytoluene | 0.4 | mg |
| Butylated Hydroxyanisole | 0.4 | mg |
| N-Methyl Pyrrolidine | to 2 | mL. |

In another embodiment there is provided a unit dose for controlling oestrus, said unit dose consisting essentially of:

| | | |
|---|---|---|
| Gonadorelin (as Acetate) BP/Ph Eur/NF | 0.10 | mg |
| Progesterone BP/Ph Eur/NF | 150.0 | mg |
| Water for Injections BP/Ph Eur/NF | 60.0 | mg |
| Phosphoric Acid 85% BP/Ph Eur/NF | 20.0 | mg |
| Butylated Hydroxytoluene | 0.4 | mg |
| Butylated Hydroxyanisole | 0.4 | mg |
| N-Methyl Pyrrolidine | to 2 | mL. |

In another embodiment there is provided a unit dose for controlling oestrus, said unit dose consisting of:

| | | |
|---|---|---|
| Gonadorelin (as Acetate) BP/Ph Eur/NF | 0.10 | mg |
| Progesterone BP/Ph Eur/NF | 150.0 | mg |
| Water for Injections BP/Ph Eur/NF | 60.0 | mg |
| Phosphoric Acid 85% BP/Ph Eur/NF | 20.0 | mg |
| Butylated Hydroxytoluene | 0.4 | mg |
| Butylated Hydroxyanisole | 0.4 | mg |
| N-Methyl Pyrrolidine | to 2 | mL |

In a third aspect of the invention there is provided a process for making a formulation for controlling oestrus comprising combining:
a solution comprising GnRH or an acceptable salt or analogue thereof dissolved in water,
a solvent, and
progesterone or an analogue thereof.

In another aspect of the invention there is provided a process for making a formulation for controlling oestrus consisting essentially of or consisting of combining:
a solution comprising GnRH or an acceptable salt or analogue thereof dissolved in water,
a solvent, and
progesterone or an analogue thereof.

In another aspect of the invention there is provided a process for making a pharmaceutically acceptable formulation for controlling oestrus comprising, consisting essentially of or consisting of combining:
a solution comprising GnRH or an acceptable salt or analogue thereof dissolved in water,
a solvent, and
progesterone or an analogue thereof.

The process may additionally comprise adding an injection aid and/or a preservative. The injection aid and/or the preservative, if present, may be added before, after or with the solvent. The step may comprise dissolving the injection aid and/or preservative, if present, in the solvent. The process may comprise agitating the formulation sufficiently to dissolve the progesterone or analogue thereof. The agitating may comprise stirring, swirling, shaking, mixing, sonicating or otherwise agitating. The carrier may be a solvent. The solvent may be a carrier.

The salt of GnRH may be GnRH acetate. One or more of the progesterone or analogue thereof, the water, the solvent, the injection aid and the preservative may be as described in the first aspect of the invention. The combining may be such that the amounts, or concentrations, of the components of the formulation are as described in the first aspect.

The process may also comprise adding a further amount of the solvent, or an amount of a second solvent, after the combining, said second solvent being miscible with the solvent added initially. The further amount may be sufficient to bring the volume of the formulation to a desired volume. It may be sufficient to bring the concentrations of the GnRH or salt or analogue thereof, and of the progesterone or analogue thereof, to desired values in the formulation. The ratio of the initial amount of the solvent to the further amount thereof, or the ratio of the amount of the solvent to the amount of the second solvent, may be between about 5:1 and about 100:1, or between about 5:1 and about 20:1, on a v/v basis.

The process may comprise the step of providing the solution comprising GnRH or an acceptable salt or analogue thereof dissolved in water. This step may comprise dissolving GnRH or a salt or analogue thereof in the water. The concentration of the GnRH or salt or analogue thereof in the solution thereof in water may be between about 0.1 and about 2% w/w or w/v, and may be about 1%.

The solvent and, if present, the second solvent, may be a solvent for the progesterone or the analogue thereof. The solvent and, if present, the second solvent, may comprise an organic solvent. The organic solvent may be a polar solvent. The organic solvent may be aprotic, or may be protic. The carrier may be sufficiently miscible with water that the formulation is homogeneous, or that the water in the formulation is dissolved in the solvent.

The combining may be such that the ratio between the GnRH or salt or analogue thereof and the progesterone or analogue thereof is between about 1:2000 and about 1:3000, or between about 1:1000 and 1:3000, for example about 1:2500 w/w or about 1:1500 w/w. The further amount of the solvent, or the amount of a second solvent, may be sufficient to bring the concentrations of GnRH (or salt or analogue thereof) and progesterone or analogue thereof to between about 0.02 and about 0.1 mg/ml and about 25 to about 150 mg/ml respectively in the formulation.

The process may comprise the step of adjusting the pH of the formulation. The adjusting may comprise adding a pH adjusting agent, e.g. an acid. The adjusting may be to a pH of between about 4 and about 6 or between about 4 and 5, 5 and 6 or 4.5 and 5.5, e.g. about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9 or 6.

The process may additionally comprise sterilising, e.g. terminally sterilising, the formulation. The sterilising may comprise filtering the formulation through a sterilising filter, such as a 0.2 micron filter or a filter of pore size less than about 0.2 microns. The filter may have an absolute pore size cutoff of about 0.2 microns or less than about 0.2 microns (e.g. 0.19, 0.18, 0.17, 0.16, 0.15, 0.14, 0.13, 0.12, 0.11 or 0.1 microns).

In an embodiment there is provided a process for making a formulation for controlling oestrus comprising, consisting essentially of or consisting of:
combining a solution comprising GnRH or an acceptable salt or analogue thereof dissolved in water, an acceptable carrier, and progesterone or an analogue thereof and optionally at least one of an injection aid and a preservative; and
adding a further amount of the solvent to form the formulation.

In another embodiment there is provided a process for making a formulation for controlling oestrus comprising, consisting essentially of or consisting of:
providing a first solution comprising GnRH acetate dissolved in water;
combining the solution, progesterone, a solvent and optionally an injection aid and/or a preservative such that the w/w ratio between the GnRH acetate and the progesterone is between about 1:2000 and about 1:3000, for example about 1:2500;
adding a further amount of the solvent to the combined first and second solutions so as to bring the concentrations of GnRH acetate and progesterone to between about 0.02 and about 0.1 mg/ml and about 100 to about 150 mg/ml respectively to form the formulation; and
sterilising the formulation by filtering through a sterilising filter.

In another embodiment there is provided a process for making a formulation for controlling oestrus comprising, consisting essentially of or consisting of:
providing a first solution comprising GnRH acetate dissolved in water;
combining the solution, progesterone, a solvent and optionally an injection aid and/or a preservative such that the w/w ratio between the GnRH acetate and the progesterone is between about 1:1000 and about 1:3000, for example about 1:1500;
adding a further amount of the solvent to the combined first and second solutions so as to bring the concentrations of GnRH acetate and progesterone to between about 0.02 and about 0.1 mg/ml and about 50 to about 150 mg/ml respectively to form the formulation; and
sterilising the formulation by filtering through a sterilising filter.

In another embodiment the process comprises combining a solution comprising GnRH or an acceptable salt or analogue thereof dissolved in water with a second solution comprising progesterone or an analogue thereof in a solvent. The progesterone may be dissolved in the solvent. The combining may comprise mixing, for example swirling, stirring or otherwise agitating, the first and second solutions together. The first solution may be added to the second solution or the second solution may be added to the first solution. The second solution may comprise the injection aid, if present. The second solution may comprise the preservative, if present. The formulation may be according to the first aspect of the invention.

The process may optionally comprise the step of providing the second solution. This step may comprise dissolving progesterone or an analogue thereof in the solvent, for example a dipolar aprotic solvent such as NMP. The step of providing the second solution may additionally comprise one or more of the steps of dispersing and/or dissolving the injection aid, if present, in the solution of progesterone and of dispersing and/or dissolving a preservative in the solution of progesterone.

The ratio of the first solution to the second solution may be between about 1:20 and about 1:200 or between about 1:5 and about 1:200. The concentration of progesterone or analogue thereof in the second solution may be between about 10 and about 40% w/w or w/v.

In another embodiment there is provided a process for making a formulation for controlling oestrus comprising, consisting essentially of or consisting of:
providing a first solution comprising GnRH or a salt or analogue thereof dissolved in water;
providing a second solution comprising progesterone or an analogue thereof in a solvent, said second solution optionally also comprising at least one of an injection aid and a preservative;
combining the first solution and the second solution; and
adding a further amount of the solvent to the combined first and second solutions to form the formulation.

In another embodiment there is provided a process for making a formulation for controlling oestrus comprising, consisting essentially of or consisting of:
providing a first solution comprising GnRH acetate dissolved in water;
providing a second solution comprising progesterone, an injection aid and a preservative in a solvent;
combining the first solution and the second solution such that the w/w ratio between the GnRH acetate and the progesterone is between about 1:2000 and about 1:3000, for example about 1:2500;
adding a further amount of the solvent to the combined first and second solutions so as to bring the concentrations of GnRH acetate and progesterone to between about 0.02 and about 0.1 mg/ml and about 100 to about 150 mg/ml respectively to form the formulation; and
sterilising the formulation by filtering through a sterilising filter.

In another embodiment there is provided a process for making a formulation for controlling oestrus comprising, consisting essentially of or consisting of:
providing a first solution comprising GnRH acetate dissolved in water;
providing a second solution comprising progesterone, an injection aid and a preservative in a solvent;
combining the first solution and the second solution such that the w/w ratio between the GnRH acetate and the progesterone is between about 1:1000 and about 1:3000, for example about 1:1500;
adding a further amount of the solvent to the combined first and second solutions so as to bring the concentrations of GnRH acetate and progesterone to between about 0.02 and about 0.1 mg/ml and about 50 to about 150 mg/ml respectively to form the formulation; and
sterilising the formulation by filtering through a sterilising filter.

In another embodiment there is provided a process for making a formulation for controlling oestrus comprising, consisting essentially of or consisting of:
providing a first solution comprising GnRH acetate dissolved in water;
providing a second solution comprising progesterone, an injection aid and a preservative in a solvent;
combining the first solution and the second solution such that the w/w ratio between the GnRH acetate and the progesterone is between about 1:1000 and about 1:3000, for example about 1:1500;
adding a further amount of the solvent to the combined first and second solutions so as to bring the concentrations of GnRH acetate and progesterone to between about 0.02 and about 0.1 mg/ml and about 50 to about 150 mg/ml respectively to form the formulation;
adding an acid so as to adjust the pH of the formulation to about 4.5; and
sterilising the formulation by filtering through a sterilising filter.

There is also provided a formulation for controlling oestrus when made by the process of the third aspect of the invention.

In a fourth aspect of the invention there is provided a process for making a unit dose for controlling oestrus in a female subject, said process comprising measuring out a quantity of a formulation comprising GnRH or an acceptable salt or analogue thereof, progesterone or an analogue thereof and an acceptable carrier, wherein said quantity is such that the unit dose comprises between about 200 and about 300 mg, or between about 150 and 300 mg, progesterone or analogue thereof and between about 0.04 and about 0.2 mg GnRH or salt or analogue thereof. In another aspect of the invention there is provided a process for making a unit dose for controlling oestrus in a female subject, said process consisting essentially of or consisting of measuring out a quantity of a formulation comprising GnRH or an acceptable salt or analogue thereof, progesterone or an analogue thereof and an acceptable carrier, wherein said quantity is such that the unit dose comprises between about 200 and about 300 mg, or between about 150 and 300 mg, progesterone or analogue thereof and between about 0.04 and about 0.2 mg GnRH or salt or analogue thereof. In another aspect of the invention there is provided a process for making a pharmaceutically acceptable or veterinarily acceptable unit dose for controlling oestrus in a female subject, said process comprising, consisting essentially of or consisting of measuring out a quantity of a formulation comprising GnRH or an acceptable salt or analogue thereof, progesterone or an analogue thereof and a pharmaceutically acceptable or veterinarily acceptable carrier, wherein said quantity is such that the unit dose comprises between about 200 and about 300 mg, or between about 150 and 300 mg, progesterone or analogue thereof and between about 0.04 and about 0.2 mg GnRH or salt or analogue thereof. The formulation may be according to the first aspect of the invention. The ratio between the GnRH or salt or analogue thereof and the progesterone or analogue thereof may be between about 1:2000 and about 1:3000, or between about 1:1000 and 1:3000, for example about 1:2500 w/w or about 1:1500 w/w. The process may comprise loading the quantity of the formulation into a container. The container may be for example a vial, a phial, an ampoule or a syringe, or some other suitable container, and may have an internal volume greater than or equal to the volume of the quantity of the formulation. The process may also comprise sealing the container. The process may also comprise sterilising the formulation before or during the loading, for example by passing the formulation through a sterilising filter.

In an embodiment there is provided a process for making a unit dose for controlling oestrus in a female subject, said process comprising loading between about 1.5 and about 4 ml of the formulation of the first aspect of the invention or of a formulation made by the process of the third aspect of the invention, into a container, for example a vial, a phial, an ampoule or a syringe, said container having a sufficient volume for the required quantity of the formulation (i.e. between at least about 1.5 and at least about 4 ml). The volume loaded into the container may contain between about 200 and about 300 mg, or between about 150 and about 300 mg, progesterone or analogue thereof and between about 0.04 and about 0.2 mg GnRH or salt or analogue thereof.

In a fifth aspect of the invention there is provided a method for increasing the number of female animals that become pregnant following a hormone intervention programme, comprising administering to said animal a unit dose of a formulation according to the present invention on day 1. In an aspect of the invention there is provided a method for increasing the number of female animals that become pregnant following a hormone intervention programme, comprising, consisting essentially of or consisting of administering to said animal a pharmaceutically acceptable or veterinarily acceptable unit dose of a formulation according to the present invention on day 1. There is also provided a method for increasing the number of female animals that become pregnant, or increasing the probability that an animal becomes pregnant, following a hormone intervention programme, comprising administering to said animal an effective amount of a formulation according to the first aspect of the invention. There is also provided a method for increasing the number of female animals that become pregnant, or increasing the probability that an animal becomes pregnant, following a hormone intervention programme, comprising, consisting essentially of or consisting of administering to said animal a pharmaceutically or veterinarily acceptable effective amount of a formulation according to the first aspect of the invention. The effective amount may be effective to increase the chance of an animal becoming pregnant during or following the programme, or following application of the method. The increase may be relative to application of a similar method in which the formulation injected at day 1 does not comprise progesterone or an analogue thereof. The method of the fifth aspect may additionally comprise administering to the animal prostaglandin, or an analogue thereof having similar biological activity, on day 7. The method may additionally comprise administering GnRH or an analogue or salt thereof on day 9. The method may comprise artificially inseminating the animal. Artificial insemination may be conducted on day 9 or 10 of the programme. Each step of administering may, independently, comprise injecting the particular substance or substances into the animal. The injection may be subdermal, intramuscular or intravenous.

The present invention also provides a formulation according to the first aspect or made by the process of the third aspect, or a unit dose according to the second aspect or made by the process of the fourth aspect, when used in a hormone intervention programme. The programme may be for increasing the number of animals that become pregnant or for increasing the probability that an animal becomes pregnant. There is also provided the use of GnRH or an acceptable salt or analogue thereof, progesterone or an analogue thereof, and an acceptable solvent for the manufacture of a stable formulation for controlling oestrus in a subject.

### BRIEF DESCRIPTION OF THE DRAWINGS

A preferred form of the present invention will now be described by way of example with reference to the accompanying drawings wherein:

FIG. 1 is a schematic diagram of follicular wave and corpus luteum stages and FSH and LH profiles for a 2-wave ovarian cycle;

FIG. 2 shows a distribution of cycle lengths (from return to service data) for Australian pasture-based dairy cows (2001);

FIG. 3 is a chart illustrating the effect of ovulation and insemination timing on fertility risk for cows receiving artificial insemination (AI);

FIG. 4 is a schematic representation of the timing between ovulation, insemination and the periods of maximum ova and sperm fertility for early and late ovulating cows submitted to the OVsynch programme described herein;

FIG. 5 is a graph showing the distribution of cow age by treatment group in example 1;

FIG. 6 is a graph showing the distribution of number of days calved by Mating Start Date (MSD) strata by treatment group in example 1; and

FIG. 7 is a graph showing the distribution of return intervals for cows not pregnant to fixed time artificial insemination (FTAI) in example 1.

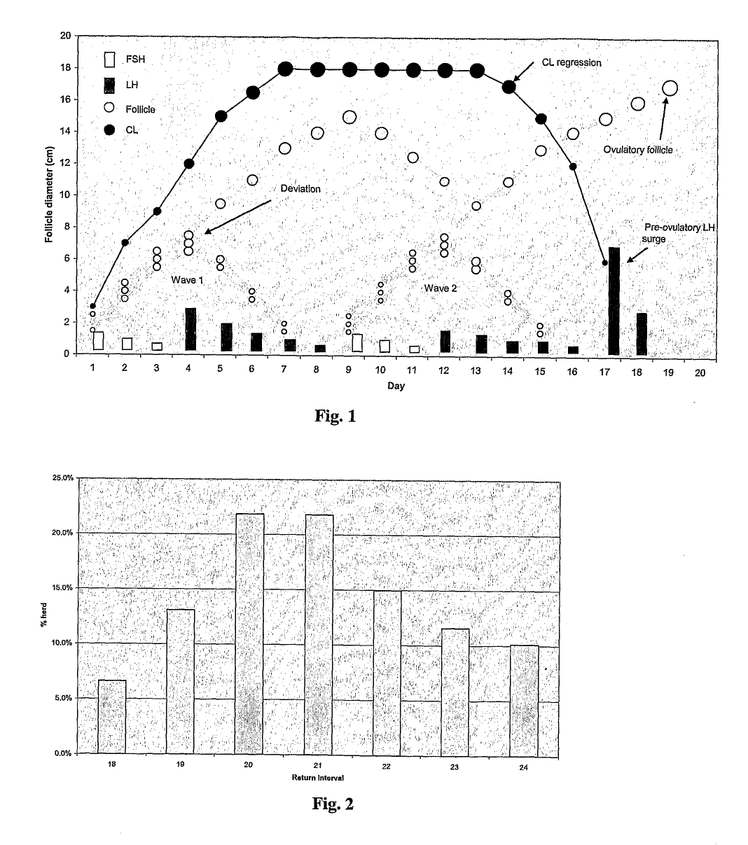

### DETAILED DESCRIPTION OF THE PREFERRED EMBODIMENTS AND OTHER EMBODIMENTS

The present inventors have discovered a novel method for increasing the number of cows that become pregnant following a hormone intervention programme. The incorporation of progesterone at the time of the first GnRH injection in the early phase of the OVsynch programme described herein, has been shown to significantly increase the pregnancy rate in dairy cows. The present invention provides a formulation for use in such a hormone intervention programme.

Accordingly the present invention provides a stable formulation for controlling oestrus in a subject comprising:
GnRH (Gonadotrophin Releasing Hormone) or an acceptable salt or analogue thereof;
progesterone or an analogue thereof; and
an acceptable carrier.

“Controlling” oestrus may refer to controlling and/or altering the time at which oestrus occurs in the subject, so that insemination (e.g. artificial insemination) may be timed so as to improve the chances of achieving pregnancy. The formulation may be used in a programme in combination with other formulations and procedures in order to achieve this goal.

It should be understood that where mention is made in this specification of GnRH, this may equally refer to an acceptable analogue or salt thereof (Gonadorelin of Gonadorelin acetate may be used), and where mention is made of progesterone, this may equally refer to an analogue thereof with similar hormonal activity. In the specification and claims the term “acceptable” may be taken to mean pharmaceutically acceptable or veterinarily acceptable. It may refer to non-toxic and/or non-irritating properties. In the specification and claims the term “analogue” may be taken to refer to a compound that is similar in chemical structure and has comparable, although not necessarily identical, pharmacological or veterinary activity. It may refer to a derivative. Each one of the formulations and unit doses of the invention may be pharmaceutically acceptable or veterinarily acceptable.

As referred to herein, an analogue of GnRH is a derivative of GnRH, which derivative comprises addition, deletion or substitution of one or more amino acids, such that the polypeptide retains substantially the same function as GnRH. It may be obtained by conservative substitution of one or more amino acids of GnRH. Conservative substitution refers to substitution or replacement of one amino acid for another amino acid with similar properties within a polypeptide chain. For example the substitution of the charged amino acid glutamic acid (Glu) for the similarly charged amino acid aspartic acid (Asp) may be a conservative amino acid substitution.

The salt or analogue of GnRH may be capable of providing short acting release qualities to the compound. These hormones are believed to work by providing a pulsatile spike and they have a very short half life in the body. Consequently longer acting release preparations may be less effective than short acting release compounds. It is desirable that the progesterone or analogue thereof is released rapidly to provide a spike of hormone at the highest concentration. The concentration of progesterone in the formulation may be between about 25 and about 150 mg/ml, or between about 50 and 150, 75 and 100, 60 and 90, 70 and 80,100 and 150, 100 and 130, 100 and 120, 120 and 150, 110 and 140 or 120 and 130 mg/ml, and may be about 25, 50, 75, 100, 105, 110, 115, 120, 125, 130, 135, 140, 145 and 150 mg/ml. In a unit dose of the formulation, there may be between about 200 and about 300 mg, or between about 220 and 300, 150 and 300, 150 and 200, 130 and 170, 140 and 160, 170 and 300, 200 and 270, 200 and 250, 200 and 220, 220 and 280, 230 and 270, 240 and 260 or 245 and 255 mg, and may be about 150, 160, 160, 180, 190, 200, 210, 220, 230, 240, 245, 250, 255, 260, 270, 280, 290 or 399 mg. It will be understood that some other compound having similar biological activity to progesterone, such as a progesterone analogue, a substituted progesterone or the like or some mixture of two or more such compounds, may be used in place of progesterone. In that case the above concentrations and amounts may be adjusted to allow for different potency or level of activity of the other compound(s). The concentration of progesterone may be effective to increase the chance of an animal becoming pregnant. It may be effective for that purpose when used in a programme as described herein, for example the OVsynch programme, relative to use of the same programme in which the formulation of GnRH has no progesterone.

The concentration of GnRH or salt or analogue thereof in the formulation may be between about 0.02 and about 0.1 mg/ml, or between about 0.05 and 0.1, 0.02 and 0.08, 0.02 and 0.05, 0.03 and 0.07, 0.04 and 0.06 or 0.045 and 0.055 mg/ml, and may be about 0.02, 0.03, 0.04, 0.045, 0.05, 0.055, 0.06, 0.07, 0.08, 0.09 or 0.1 mg/ml. In a unit dose of the formulation there may be between about 0.04 and about 0.2 mg, or between about 0.05 and 0.2, 0.1 and 0.2, 0.04 and 0.15, 0.04 and 0.1, 0.04 and 0.07, 0.05 and 0.15, 0.07 and 0.13, 0.08 and 0.12 or 0.09 and 0.11 mg. It will be understood that some other compound having similar biological activity to GnRH, such as a GnRH salt or analogue or a mixture of two or more of such salts or analogues, may be used in place of GnRH. In that case the above concentrations and amounts may be adjusted to allow for different potency or level of activity of the other compound(s).

The w/w ratio of GnRH or salt or analogue thereof to progesterone or analogue thereof in the formulation may be between about 1:2000 and about 1:3000, or between about 1:1000 and 1:3000, is 1:1000 and 1:2000, 1:2000 and 1:2500, 1:2500 and 1:3000, 1:2000 and 1:2700, 1:2300 and 1:3000, 1:2200 and 1:2800, 1:2300 and 1:2700, 1:2400 and 1:2600, 1:2450 and 1:2550 or 1:2490 and 1:2510, for example about 1:1000. 1:1100, 1:1200, 1:1300, 1:1400, 1:1500, 1:1600, 1:1700, 1:1800, 1:1900, 1:2000, 1:2100, 1:2200, 1:2300, 1:2400, 1:2450, 1:2460, 1:2470, 1:2480, 1:2490, 1:2500, 1:2510, 1:2520, 1:2530, 1:2540, 1:2550, 1:2600, 1:2700, 1:2800, 1:2900 or 1:3000. The ratio may be such that the formulation, when injected into a subject in the early phase of an OVsynch programme, as described herein, provides a greater chance of pregnancy in the subject than if no progesterone or analogue thereof were present in the formulation, or than if an alternative formulation were injected having the same level of GnRH or salt or analogue thereof as the formulation of the invention but with no progesterone or analogue thereof.

The carrier may comprise a solvent, and may comprise water. The water may be suitable for injection. It may be present at between about 2 and about 50 mg/ml in the formulation, or between about 2, and 40, 2 and 30, 2 and 20, 2 and 10, 10 and 50, 20 and 50, 25 and 50, 30 and 50, 10 and 40 or 10 and 30 mg/ml, for example about 2, 3, 4, 5, 6, 7, 8, 9 10, 15, 20, 25, 30, 35, 40, 45 or 50 mg/ml. A unit dose may comprise between about 4 and about 100 mg water, or between about 10 and 100, 25 and 100, 50 and 100, 2 and 50, 2 and 25, 2 and 10, 10 and 50 or 20 and 50 mg water, and may comprise about 4, 5, 6, 7, 8, 9, 10, 15, 20, 25, 30, 35, 40, 45, 50, 55, 60, 65, 70, 75, 80, 85, 90, 95 or 100 mg water, or may comprise more than about 100 mg water.

The solvent may be capable of dissolving progesterone or analogue thereof and may be capable of forming a solution of progesterone or the analogue thereof that is 40% w/v progesterone or analogue, or 35, 30, 25, 20, 19, 18, 17, 16, 15, 14, 13, 12, 11 or 10% progesterone or analogue. The solvent may be mixed with (or may comprise) one or more cosolvents in order to achieve the appropriate solvency. The solvent and the one or more cosolvents should be at least partially miscible. Suitable solvents and cosolvents include polar organic solvents, e.g. dipolar aprotic solvents such as N-methyl-2-pyrrolidone (NMP), glycerol formal and dimethylsulfoxide (DMSO) and mixtures of any two or more of these. A suitable cosolvent may be water. In the formulation of the present invention the progesterone or analogue thereof is dissolved in a partially hydrophobic excipient and the GnRH, or salt or analogue thereof, is dissolved in a small amount of water which the carefully selected partially hydrophobic excipient can tolerate. Extremely hydrophobic excipients such as chloroform are not capable of tolerating sufficient water although they may be capable of dissolving a large amount of progesterone. At the other extreme, progesterone is not soluble in water. Consequently an excipient (carrier) has to be carefully selected. A suitable solvent for use in the invention may be one with non-polar groups to dissolve or associate with the progesterone or analogue thereof and polar groups to enable the solvent to tolerate a small amount of water is (containing the GnRH or salt or analogue thereof). Two solvents that may be used in the present invention are NMP and glycerol formal. If the solvent is too hydrophobic, the water cannot be added without separating. If the solvent is too hydrophilic the progesterone or analogue thereof will not be soluble at the high concentration required. Solvents may be used in combination.

The formulation may also comprise an injection aid. The injection aid may be present between about 10 and about 50 mg/ml, or between about 10 and 40, 10 and 30, 10 and 20, 20 and 50, 30 and 50, 20 and 40, 20 and 30 or 23 and 27 mg/ml, and may be present at about 10, 15, 20, 21, 22, 23, 24, 25, 26, 27, 28, 29, 30, 35, 40, 45 or 50 mg/ml. In a unit dose of the formulation there may be between about 20 and 100 mg or the injection aid, or between about 40 and 100, 60 and 100, 20 and 80, 20 and 60, 30 and 70, 40 and 60 or 45 and 55 mg, and may be about 20, 25, 30, 35, 40, 45, 50, 55, 60, 65, 70, 75, 80, 85, 90, 95 or 100 mg. The injection aid may be at least partially soluble in the solvent and/or in a mixture of the solvent with the cosolvents. The injection aid may be polymeric or monomeric or oligomeric. The injection aid may be a surfactant. It may be a hydrophilic polymer, and may be a water soluble polymer. It may be for example polyvinylpyrrolidone, or polyethylene oxide, a carboxypolymethylene polymer or some other suitable hydrophilic polymer, or may be a mixture of these. The injection aid may be at least partially soluble in the carrier, or in the solvent. The injection aid may be a compound, or mixture of compounds, that facilitates injection of the formulation (or unit dose) into an animal. It may function as a lubricant during injection into the animal. The formulation may contain no injection aid.

The formulation may optionally also comprise one or more preservatives, although in some embodiments, no preservative is present. If a preservative is present, it may be in a concentration sufficient to prevent microbial growth within the formulation. It may prevent microbial growth for at least about 3 months, or at least about 6, 9, 12, 15, 18, 21, 24, 30 or 36 months. Thus the formulation, once terminally sterilised and stored in a sealed container, may have a shelf life of at least about 6, 9, 12, 15, 18, 21, 24, 30 or 36 months, and may have a shelf life of about 6, 9, 12, 15, 18, 21, 24, 30 or 36 months. The preservative may be present in the formulation between about 0 and about 50 mg/ml, or between about 0 and 20, 0 and 10, 0 and 5, 5 and 50, 5 and 20, 1 and 5, 5 and 15, 5 and 10, 10 and 20, 8 and 12 or 9 and 11 mg/ml, and may be present at about 0.5, 1, 2, 3, 4, 5, 6, 7, 8, 9, 10, 11, 12, 13, 1,4 15, 16, 17, 18, 19, 20, 25, 30, 35, 40, 45 or 50 mg/ml, or may have a different concentration, depending on the nature of the preservative. A unit dose of the formulation may comprise between about 0 and about 100 mg of the preservative, or between about 0 and 50, 0 and 20, 0 and 10, 0.5 and 100, 1 and 100, 5 and 100, 10 and 100, 20 and 100, 50 and 100, 0.5 and 50, 1 and 50, 1 and 30, 1 and 20, 1 and 10, 5 and 50, 5 and 20, 10 and 20, 10 and 40, and 30, 20 and 50, 30 and 50, 15 and 30 or 15 and 25 mg, and may comprise about 0.1, 0.2, 0.3, 0.4, 0.5, 0.6, 0.7, 0.8, 0.9, 1, 2, 3, 4, 5, 10, 15, 20, 25, 30, 35, 40, 45, 50, 60, 70, 80, 90 or 100 mg of the preservative, or may comprise some other amount. The preservative may be at least partially soluble in the solvent and/or in a mixture of the solvent with the cosolvents. The preservative may comprise a mixture of two or more preservative compounds. The preservative may be, or comprise, an antioxidant or more than one antioxidants. Suitable antioxidants include butylated hydroxytoluene (BHT), butylated hydroxyanisole (BHA), propyl gallate (PGal), mono and bi-sulfites, ascorbates, cysteine, sulfoxalates, gentistic acid, glutamates, thyoglycolate, thioglycerol and mixtures of any two or more thereof.

The formulation of the present invention is a stable formulation. It may be stable upon storage at ambient and elevated temperatures. It may be stable for a period of at least 3 months, or at least 6, 9, 12, 15, 18, 21, 24, 27, 30, 33 or 36 months. It may be stable for that period at a temperature below about 40° C., or below about 35, 30, 25 or 20° C., or between about 5 and about 40° C., or between about 5 and 30, 5 and 25, 5 and 20, 10 and 40, 20 and 40, 30 and 40, 10 and 25, and 30, 15 and 25 or 20 and 30° C., and may be stable for that period at about 5, 10, 15, 20, 25, 30, 35 or 40° C. It may be stable at less than 20° C. In this context, “stable” may refer to the condition in which the GnRH or salt thereof, and the progesterone or analogue thereof, do not separate from the formulation, and/or to the condition in which the concentrations of the GnRH or salt or analogue thereof, and the progesterone or analogue thereof, in the formulation do not vary by more than about 10% from their initial values, and/or to the condition in which the formulation remains homogeneous. The formulation may be stable with respect to degradation of the GnRH and/or the progesterone. The formulation may be stable to radiation at 254 nm. It may be stable for at least about 6 days, or 5 days, or 4 days under radiation at 254 nm. In this context “stable” may refer to a reduction in GnRH concentration of less than about 15%, or less than about 14, 13, 12, 11, 10, 9, 8, 7, 6, 5, 4, 3, 2, 1 or 0.5% or between 0.01 and 15%/day. The rate of loss of GnRH under irradiation at 254 nm may be less than about 3%/day, or less than about 2.5 or 2%/day, or between about 0 and 3, 0 and 2.5, 0 and 2, 0 and 1.9, 0 and 1.8, 0 and 1.7, 0 and 1.6 or 0 and 1.5%/day, 0.01 and 3, 0.01 and 2.5, 0.01 and 2, 0.01 and 1.9, 0.01 and 1.8, 0.01 and 1.7, 0.01 and 1.6, 0.01 and 1.5, 0.01 and 1.4, 0.01 and 1.3, 0.01 and 1.2, 0.01 and 1.1, 0.01 and 1.0, 0.01 and 0.9, 0.01 and 0.8, 0.1 and 3, 0.1 and 2.5, 0.1 and 2, 0.1 and 1.9, 0.1 and 1.8, 0.1 and 1.7, 0.1 and 1.6, 0.1 and 1.5, 0.1 and 1.4, 0.1 and 1.3, 0.1 and 1.2, 0.1 and 1.1, 0.1 and 1.0, 0.1 and 0.9, 0.1 and 0.8, 0.5 and 3, 0.5 and 2.5, 0.5 and 2, 0.5 and 1.9, 0.5 and 1.8, 0.5 and 1.7, 0.5 and 1.6, 0.5 and 1.5, 0.5 and 1.4, 0.5 and 1.3, 0.5 and 1.2, 0.5 and 1.1, 0.5 and 1.0, 0.5 and 0.9, 0.5 and 0.8, 0.5 and 0.7%/day, e.g. about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3%/day. The symbol % may refer to wt %, mole % or absorbance %. The radiation for determining the above stability may for example be obtained by a Spectraline ENF-260C lamp: 365/254 nm, fitted with two 6-watt tubes.

The formulation may be acidic. It may have a pH of between about 4 and about 6 or between about 4 and 5, 5 and 6 or 4.5 and 5.5, e.g. about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9 or 6.

The progesterone or analogue thereof and the GnRH or salt or analogue thereof, and, if present, the injection aid and the preservative, may be in a form such that they are not be removable by filtration through a sterilising filter, for example a 0.2 micron filter, or a 0.22 micron filter or a 0.1 micron filter. Each may, independently, be present in solution, or in droplets or micelles of diameter less than about 0.2 microns, or less than about 0.22, 0.15, 0.1, 0.05 or 0.01 microns. The formulation of the invention may be a solution, or may be a microemulsion, a miniemulsion or a small particle size emulsion. The term microemulsion refers to an emulsion in which the dispersed phase has an average droplet size of less than 100 nm, for example 30 to 100 nm or 50 to 100 nm and which is thermodynamically stable. The term miniemulsion or small particle emulsion refers to an emulsion whose disperse phase has an average droplet size of from 100 nm to 1 μm, for example from 100 to 750 nm, 100 to 500 nm, or 100 to 200 nm. Commonly a miniemulsion will have an average droplet size of less than about 1 micron. If the formulation is not a solution, the droplet size (e.g. mean droplet size) of the formulation may be less than about 0.2 microns, or less than about 0.18, 0.16, 0.14, 0.12, 0.1, 0.08, 0.06, 0.04 or 0.02 microns, or between about 0.01 and 0.2, 0.05 and 0.2, 0.1 and 0.2, 0.01 and 0.1, 0.01 and 0.05 or 0.05 and 0.15 microns. In this case, the droplet size (mean or maximum) may be about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19 or 0.2 microns. The formulations of the invention may comprise a pharmaceutically or veterinarily acceptable surfactant. The surfactant may stabilise the formulation.

The present invention provides a unit dose of the formulation described above. The unit dose may have a total volume of between 1 and 4 ml, or may have a volume greater than 4 ml, for example 5, 10 or 20 ml. It may have a unit volume such that the quantity of the carrier in the unit dose is a non-toxic quantity to the subject when injected. The volume may be between about 1 and 3, 1 and 2, 2 and 3, 1.5 and 2.5 or 1.8 and 2.2, and may be about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9 or 4 ml. The unit volume may be sufficient to deliver between about 0.04 and about 0.2 mg GnRH or an acceptable salt or analogue thereof and between about 200 and about 300 mg (or between about 150 and about 300 mg) progesterone or analogue thereof to a subject when the unit dose is injected is into the subject. The unit dose may comprise between about 0.05 and 0.2, 0.1 and 0.2, 0.04 and 1, 0.05 and 0.15 or 0.08 and 0.12 mg, and may comprise about 0.04, 0.05, 0.06, 0.07, 0.08, 0.09, 0.1, 0.11, 0.12, 0.13, 0.14, 0.15, 0.16, 0.17, 0.18, 0.19 or 0.2 mg GnRH or an acceptable salt or analogue thereof. The unit dose may comprise between about 100 and 300, 100 and 200, 150 and 300, 150 and 200, 200 and 250, 250 and 300, 220 and 280, 230 and 270 or 240 and 260 mg progesterone or analogue thereof, and may comprise about 100, 110, 120, 130, 140, 150, 160, 170, 180, 190, 200, 210, 220, 230, 240, 250, 260, 270, 280, 290 or 300 mg progesterone or analogue thereof.

The present formulation may be made by combining a solution comprising GnRH or an acceptable salt or analogue thereof dissolved in water, a solvent, and progesterone or an analogue thereof. The process may additionally comprise adding an injection aid and/or a preservative. These components may be combined be such that the amounts, or concentrations, of the components of the formulation are as described in the first aspect. The process may also comprise agitating the components during or after the combining. The agitation may comprise stirring, swirling, shaking, sonicating, mixing or some other form of agitation, and may comprise more than one of these. It will be understood that the combining may be conducted in any desired order. For example the solution of GnRH or an acceptable salt or analogue thereof may be added to the solvent and then the progesterone or analogue thereof added, followed by the injection aid and the preservative, or the solution of GnRH or an acceptable salt or analogue thereof may be added to the solvent and the progesterone or analogue thereof added, followed by the injection aid and the preservative, or the injection aid and the preservative may be added to the solvent, and then added to the solution of GnRH or an acceptable salt or analogue thereof and finally the progesterone or analogue thereof added. Many other orders of addition may be envisaged by those skilled in the art. It will also be understood that agitation, as described above, may be conducted at intermediate stages of the process. For example, the solution of GnRH or an acceptable salt or analogue thereof may be combined with the solvent, progesterone added, and agitation performed. Other components (e.g. preservative, injection aid) may then be added and further agitation performed. Thus there may be more than one (e.g. 2, 3, 4 or 5) agitation steps, and they may be the same or different.

In one example of a process, GnRH or an acceptable salt or analogue thereof is dissolved in water, combined with a solvent to form a first mixture. The first mixture may be agitated in order to homogenise it. Progesterone or analogue thereof is then added to the first mixture to form a second mixture. The second mixture is then agitated to dissolve the progesterone. Other component(s), e.g. preservative and/or injection aid, are then added to form a third mixture, which may then be agitated to dissolve the other component(s). Further solvent may be added to bring the concentrations of GnRH or acceptable salt or analogue thereof and of progesterone or analogue thereof to the required values.

The process may also comprise adding a further amount of the solvent, or an amount of a second solvent, after the combining, in order to bring the volume of the formulation to a desired volume. It may be sufficient to bring the concentrations of the GnRH or salt or analogue thereof, and of the progesterone or analogue thereof, to desired values in the formulation. The ratio of the initial amount of the solvent to the further amount thereof, or the ratio of the amount of the solvent to the amount of the second solvent, may be between about 5:1 and about 100:1, or between about 5:1 and about 20:1, on a v/v basis. It may be between about 5:1 and 50:1, 5:1 and 20:1, 5:1 and 10:1, 10:1 and 100:1, 20:1 and 100:1, 50:1 and 100:1, 10:1 and 50:1 or 20:1 and 50:1. It may be for example about 5:1, 6:1, 7:1, 8:1, 9:1, 10:1, 15:1, 20:1, 25:1, 30:1, 35:1, 40:1, 45:1, 50:1, 60:1, 70:1, 80:1, 90:1 or 100:1, or some other suitable ratio.

The process may comprise the step of providing the solution comprising GnRH or an acceptable salt or analogue thereof dissolved in water. This step may comprise dissolving GnRH or a salt or analogue thereof in the water. The concentration of the GnRH or salt or analogue thereof in the solution thereof in water may be between about 0.1 and about 2% w/w or w/v, or between about 0.2 and 2, 0.5 and 2, 1 and 2, 1.5 and 2, 0.2 and 1, 0.2 and 0.5, 0.5 and 1.5 or 0.8 and 1.2. It may be about 0.2, 0.3, 0.4, 0.5, 0.6, 0.7, 0.8, 0.9, 1, 1.1, 1.2, 1.3, 1.4, 1.5, 1.6, 1.7, 1.8, 1.9 or 2%.

In preparing the formulation, it may be convenient to prepare a first solution comprising GnRH or a salt or analogue thereof in water and a second solution comprising progesterone or an analogue thereof, and optionally the injection aid and/or the preservative, in a solvent, optionally in the presence of one or more cosolvents. The first and second solutions are then mixed, and may then be diluted with the solvent, or with a second solvent, in order to achieve the desired concentration of ingredients (GnRH or salt or analogue thereof and progesterone or analogue thereof). In preparing the first solution the GnRH or salt or analogue thereof may be added to water in a ratio of between about 0.2 and about 2%, or between about 0.2 and 1.5, 0.2 and 1, 0.2 and 0.5, 0.5 and 2, 1 and 2, 0.5 and 1.5 or 0.8 and 1.2% w/w or w/v. The ratio may be about 0.2, 0.3, 0.4, 0.5, 0.6, 0.7, 0.8, 0.9, 1, 1.1, 1.2, 1.3, 1.4, 1.5, 1.6, 1.7, 1.8, 1.9 or 2%. The ratio may be some other ratio. The ratio may be such that the formulation has a concentration of GnRH or salt or analogue thereof as described earlier. In order to dissolve the GnRH or salt or analogue thereof in the water, it may be convenient to agitate, for example stir, swirl, mix, shake or sonicate the GnRH or salt or analogue thereof with the water. In preparing the second solution, the progesterone or analogue thereof may first be dissolved in the solvent and, if required, the injection aid and/or the preservative dissolved in the resulting solution. The second solution may have between about 5 and about 50% progesterone or analogue thereof w/v, or between about 10 and 50, 12.5 and 50, and 50, 10 and 30, 10 and 20, 15 and 40, 15 and 30, 15 and 20, 20 and 50, 30 and 50, 20 and 40, 20 and 30 or 30 and 40%, and may have about 10, 12.5, 15, 20, 25, 30, 35, 40, 45 or 50% progesterone or analogue thereof w/v. It will be understood that the order of addition of the progesterone or analogue thereof and any other components of the second solution may be varied without affecting the nature of the second solution. After each addition, the mixture of carrier and components may be agitated, for example stirred, swirled, mixed, shaken or sonicated in order to promote dissolution.

The first and second solutions may be combined in a ratio such that the w/w ratio of GnRH (or salt or analogue thereof) to progesterone (or analogue thereof) is between about 1:1000 and about 1:3000, or between about 1:1000 and 1:2000, or between about 1:2000 and about 1:3000, or between about 1:2000 and 1:2500, 1:2500 and 1:3000, 1:2000 and 1:2700, 1:2300 and 1:3000, 1:2200 and 1:2800, 1:2300 and 1:2700, 1:2400 and 1:2600, 1:2450 and 1:2550 or 1:2490 and 1:2510, for example about 1:1000, 1:1100, 1:1300, 1:1400, 1:1500, 1:1600, 1:1700, 1:1800, 1:1900, 1:2000, 1:2100, 1:2200, 1:2300, 1:2400, 1:2450, 1:2460, 1:2470, 1:2480, 1:2490, 1:2500, 1:2510, 1:2520, 1:2530, 1:2540, 1:2550, 1:2600, 1:2700, 1:2800, 1:2900 or 1:3000. In order to achieve this, the ratio of the first solution to the second solution may be between about 1:20 and about 1:200 or between about 1:5 and about 1:200, or between about 1:5 and 1:100, 1:5 and 1:50, 1:5 and 1:20, 1:5 and 1:10, 1:10 and 1:200, 1:50 and 1:200, 1:100 and 1:200, 1:20 and 1:100 1:20 and 1:50 or 1:50 and 1:100, e.g. about 1:5, 1:6, 1:7, 1:8, 1:9, 1:10, 1:15, 1:20, 1:25, 1:30, 1:35, 1:50, 1:45, 1:50, 1:60, 1:70, 1:80, 1:90, 1:100, 1:110, 1:120, 1:130, 1:140, 1:150, 1:160, 1:170, 1:180, 1:190 or 1:200. The combined first and second solutions may then be diluted with the solvent, or with a second solvent capable of maintaining the components homogeneously dispersed (and/or dissolved). The additional amount of solvent, or the amount of the second solvent, may be sufficient to render the final concentrations of GnRH or salt or analogue thereof and progesterone or analogue thereof in the formulation within the ranges described earlier for the formulation of the invention, i.e. GnRH (or salt or analogue thereof) and progesterone (or analogue thereof) about 0.02 and about 0.1 mg/ml and about 25 to about 150 mg/ml respectively. Following addition of the additional amount of solvent, or the second solvent, the formulation may be then homogenised, e.g. mixed, swirled, shaken or otherwise agitated.

An alternate process for making the formulation comprises combining a solution of GnRH or an acceptable salt or analogue thereof in water, progesterone or an analogue thereof and a solvent. The process may comprise adding additional solvent. The solution of GnRH or acceptable salt or analogue thereof may be the same as described above.

The formulation may comprise, consist essentially of or consist of:

| | | |
|---|---|---|
| Gonadorelin (as Acetate) BP/Ph Eur/NF | 0.025-0.075 | mg/ml |
| Progesterone BP/Ph Eur/NF | 60-90 | mg/ml |
| Water for Injections BP/Ph Eur/NF | 20-40 | mg/ml |
| Phosphoric Acid 85% BP/Ph Eur/NF | 5-15 | mg/ml |
| Butylated Hydroxytoluene | 0.1-0.3 | mg/ml |
| Butylated Hydroxyanisole | 0.1-0.3 | mg/ml |

| | |
|---|---|
| N-Methyl Pyrrolidine | to 100%. |

The formulation may comprise, consist essentially of or consist of:

| | | |
|---|---|---|
| Gonadorelin (as Acetate) BP/Ph Eur/NF | 0.04-0.06 | mg/ml |
| Progesterone BP/Ph Eur/NF | 70-80 | mg/ml |
| Water for Injections BP/Ph Eur/NF | 25-35 | mg/ml |
| Phosphoric Acid 85% BP/Ph Eur/NF | 7.5-12.5 | mg/ml |
| Butylated Hydroxytoluene | 0.175-0.225 | mg/ml |
| Butylated Hydroxyanisole | 0.175-0.225 | mg/ml |

| | |
|---|---|
| N-Methyl Pyrrolidine | to 100% |

A unit dose of the formulation may comprise, consist essentially of or consist of:

| | | |
|---|---|---|
| Gonadorelin (as Acetate) BP/Ph Eur/NF | 0.05-0.20 | mg |
| Progesterone BP/Ph Eur/NF | 125-175 | mg |
| Water for Injections BP/Ph Eur/NF | 40-80 | mg |
| Phosphoric Acid 85% BP/Ph Eur/NF | 10-30 | mg |
| Butylated Hydroxytoluene | 0.2-0.6 | mg |
| Butylated Hydroxyanisole | 0.2-0.6 | mg |
| N-Methyl Pyrrolidine | to 1.5-2.5 | mL |

A unit dose of the formulation may comprise, consist essentially of or consist of:

| | | |
|---|---|---|
| Gonadorelin (as Acetate) BP/Ph Eur/NF | 0.075-0.175 | mg |
| Progesterone BP/Ph Eur/NF | 140-160 | mg |
| Water for Injections BP/Ph Eur/NF | 50-70 | mg |
| Phosphoric Acid 85% BP/Ph Eur/NF | 15-25 | mg |
| Butylated Hydroxytoluene | 0.3-0.5 | mg |
| Butylated Hydroxyanisole | 0.3-0.5 | mg |
| N-Methyl Pyrrolidine | to 1.75-2.25 | mL |

The formulation may be used in a programme to control oestrus in a subject. The programme may involve injecting the subject with an effective amount of the formulation (e.g. with a unit dose) on day 1. Additional steps of the programme may include injecting the subject with prostaglandin or an analogue thereof on day 7, injecting the subject with GnRH or a salt or analogue thereof on day 9 and artificial insemination of the subject on day 9 or 10. The days are numbered from the commencement of the programme. The effective amount may be effective to increase the chance of an animal becoming pregnant during or following the programme. The programme may increase the number of animals that become pregnant following a hormone intervention programme. It may increase the chance relative to use of a similar programme in which the formulation injected on day 1 is replaced by a formulation having no progesterone, or analogue thereof. Thus in use a unit dose of the formulation may be injected into the subject. Seven days following the injection, a prostaglandin injection may be given to the subject. Nine days after the initial injection, a GnRH (or salt or analogue thereof) injection may be given to the subject. On the same or the following day the subject may be inseminated.

The formulation of the present invention is suitable for controlling oestrus in a subject. The subject may be a vertebrate, and may be a domestic vertebrate. The subject may be a female subject. The vertebrate may be a mammal, a marsupial or a reptile. The mammal may be a primate or non-human primate or other non-human mammal. The mammal may be selected from the group consisting of human, non-human primate, equine, murine, bovine, leporine, porcine, ovine, caprine, feline and canine. The mammal may be selected from a human, horse, cattle, buffalo, sheep, pig, dog, cat, goat, llama, rabbit and a camel, for example. The subject may be a cow, for example a mature cow or a heifer.

### Hormonal Control System

To develop hormone intervention schemes, it is vital that the action of each hormone is understood and the interaction or control of that hormone action by other hormones is clearly understood. This is very complex as illustrated below.

| Hormone | Source | Target | Action | Regulates | Regulated by |
|---|---|---|---|---|---|
| GnRH | Hypothalamus | Pituitary | Controls | FSH and LH | Progesterone |
| | | | release of | levels | and |
| | | | FSH and LH | | Oestrogen |
| | | | by the | | (negative |
| | | | pituitary | | feedback) |
| FSH | Pituitary | Ovary (follicles) | Promotes | Follicular | GnRH and |
| | | | recruitment, | wave timing | Inhibin |
| | | | growth and | and | (negative |
| | | | oestrogen | oestrogen | feedback) |
| | | | production by | levels | |
| | | | follicles | | |
| LH | Pituitary | Ovary (dominant | Provides final | Follicular | GnRH |
| | | follicle, and CL) | boost to | maturation, | |
| | | | dominant | ovulation and | |
| | | | follicle | CL lifespan | |
| | | | growth. | and activity | |
| | | | Initiates | | |
| | | | ovulation. | | |
| | | | Maintains the | | |
| | | | CL | | |
| Oestrogen | Ovarian follicles | Hypothalamus, | Initiates | Oestrus, | FSH, LH and |
| | | reproductive tract | oestrus | GnRH | GnRH |
| | | (uterus etc), | behaviour, | release, | |
| | | udder | prepares RT | | |
| | | | for | | |
| | | | conception, | | |
| | | | helps initiate | | |
| | | | lactation | | |
| Progesterone | Corpus luteum | Hypothalamus, | Prevents | Cycle length, | LH |
| (P4) | | reproductive tract | oestrus, | GnRH | |
| | | (uterus etc), | maintains | release, | |
| | | udder | uterine | pregnancy, | |
| | | | environment | lactation | |
| | | | for | | |
| | | | pregnancy, | | |
| | | | helps initiate | | |
| | | | and maintain | | |
| | | | lactation | | |
| Inhibin | Follicle | Hypothalamus, | Controls | Early follicular | FSH |
| | | udder | follicle | wave | |
| | | | recruitment | development | |
| | | | but not late | | |
| | | | stage | | |
| | | | development | | |

### Hormones in Detail

### GnRH

Exogenous GnRH-induced LH surges have been recorded to have a duration of 7-14 hours duration with peak serum levels of 9-22 ng/mL. Spontaneously ovulating cows generally have an LH surge of 9-17 hours duration and a peak serum magnitude of 5-42 ng/mL. The timing of peak LH levels has been found to be identical between natural GnRH release and exogenous GnRH groups.

The GnRH release associated with the pre-ovulatory LH surge is typically around 4-8 hours after the start of oestrus and the subsequent LH surge occurs typically around one hour after GnRH release. This tends to time ovulation to within an eight-hour window between 24 to 32 hours after GnRH release.

Administration of exogenous GnRH between 11 and 12 days after insemination has been demonstrated to produce an increase in pregnancy rate. It is thought the extra GnRH recruits extra (accessory) corpora lutea from the dominant follicles of the subsequent follicular wave. However, accessory CLs appear to only be recruited if the dominant follicle is on the ovary that is on the same side as the pregnancy horn. This may be necessary to negate the local effects of prostaglandin release from the non-gravid horn of the uterus and introduces yet another variable to be addressed in hormone intervention programmes.

GnRH does not reliably ovulate the largest follicle when applied to maiden heifers. This may be due to the fact that heifers do not have a functionally dominant follicle (i.e. one that is as receptive to LH) for as long a period as cows. These observations have prevented recommendations for the use of the OVsynch protocol in this class of animals, however, FTAI conception rates approaching those of cows have been obtained from use of the OVsynch protocol in heifers.

### LH

The follicle that becomes dominant develops LH receptors at around the second day of the wave. These become highly active by around day 4 of the wave. Follicles therefore are responsive to LH tropism from days 4-8 of the wave growth cycle. Follicles<day 4 are not big enough to respond to LH and follicles>8 days are post-dominant and regressing (the new follicular wave has already started).

Follicle growth beyond deviation is LH controlled. LH pulses occur at around every two hours and at low levels. In earlier work it was found that 50 μg of LH injected intravenously mimicked endogenous LH release. This is further supported by evidence to suggest that post-deviation follicular growth can be stimulated by administration of small amounts of LH in a pulsatile manner. The amount of LH stimulation received after deviation therefore determines the final size of the ovulatory follicle, the likelihood of ovulation occurring and the likelihood of formation of a functional and adequate CL.

The LH surge that initiates ovulation typically occurs around 8 hours after the start of oestrous. In general the surge lasts 9-17 hours and produces a peak LH level between 5 and 42 ng/mL. Ovulation occurs at around 24-32 hours after LH surge. This has implications for fertilisation as ova are most fertile from ovulation to around 6-12 hours after ovulation. Fertility declines as the unfertilised ova ages beyond this time. Survival of the embryo decreases as the pre-fertilisation oocyte age increases after ovulation. It is therefore important that sperm be present within the oviduct at the time of ovulation. Mating programmes involving insemination after the time of ovulation result in an aged oocyte being present for fertilisation—these oocytes have reduced fertility. Timing of the artificial insemination with regard to the age of the ova is critical for optimal fertility.

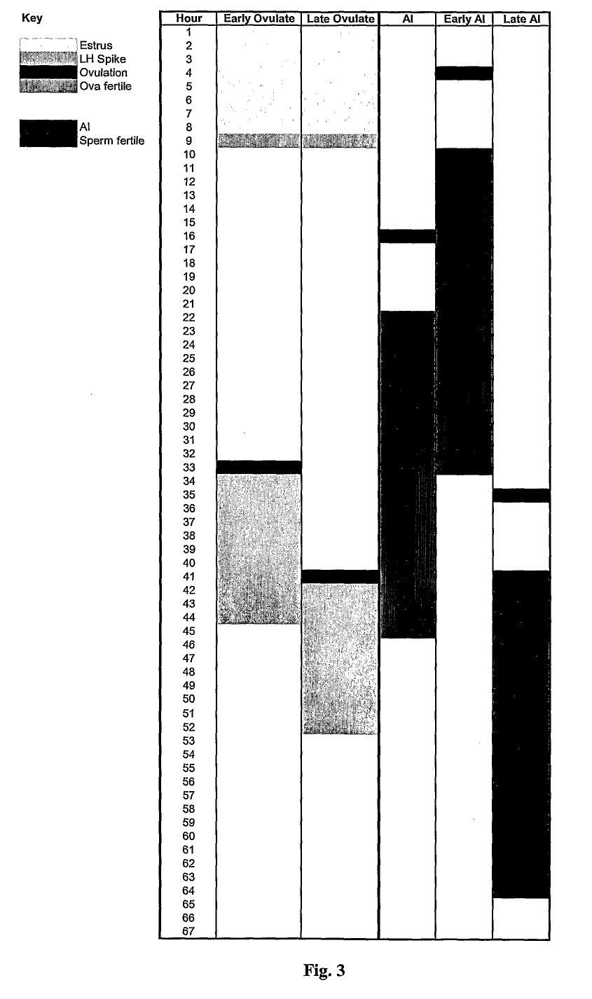

Sperm must undergo capacitisation once within the female reproductive tract. This is a process whereby they become capable of fertilisation. The sperm must also be transported to the oviduct where fertilisation must occur. The process of capacitisation and transport takes between 2-6 hours. Sperm are most fertile within the first 24-hour period within the female tract (after capacitisation). Frozen sperm have a shorter period of fertility than fresh sperm and differences between sires in fertility exist. These differences can become apparent when cows are inseminated at incorrect times (eg after ovulation). The current industry recommendation is that cows be inseminated at the first opportunity after being detected on heat. This may result in less viable sperm present at ovulation when semen from poorly fertile bulls is used in cows inseminated whilst in the early stages of oestrus, but it will avoid the risk of delayed fertilisation of ova arising from late insemination because aged ova have reduced fertility. For many farms that practice once-per-day artificial insemination (AI) this will mean insemination the following day. However, farms that utilise twice-per-day insemination may obtain a small advantage presenting as a slight increase in AI conception rates. The effect of early and late insemination on sperm and ova viability at fertilisation can be gauged from examination of FIG. 3. The combined effect of a reduced period of fertility for frozen sperm and delayed fertilisation of (aged) oocytes for cows that ovulate early after the LH spike can be a reduction in herd conception rate. Systems that time both ovulation and AI may result in better synchrony between ova and sperm within the female tract.

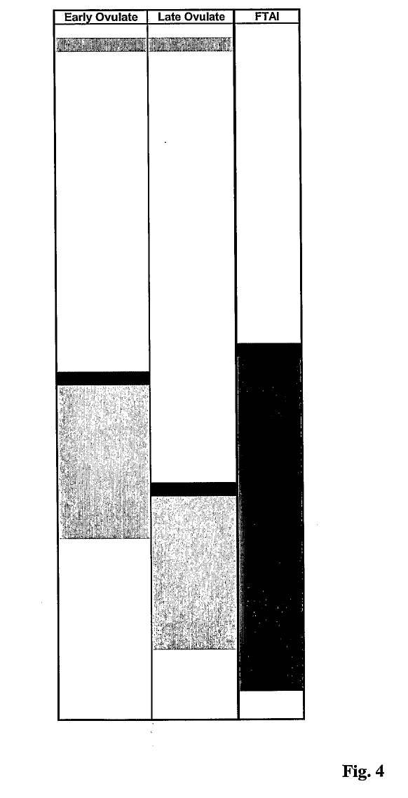

Programmes that eliminate the need for heat detection have become more popular. These programmes eliminate the problems of timing insemination with respect to ovulation and of detecting all cows on heat. The timing between ovulation, insemination and the window of fertility for ova and sperm for the OVsynch programme is presented in FIG. 4.

The second follicular wave within an ovarian cycle is typically associated with the production of small follicles. This is thought to be due to a reduction in LH stimulus for the post-deviation follicle. There are reduced levels of circulating LH during the second follicular wave because of concurrent elevated levels of progesterone arising from the active CL. It has been proposed that two-wave cows may have reduced fertility due to this effect. Three-wave cows typically produce a larger ovulatory follicle in the third wave of the cycle because the CL undergoes regression at an earlier stage of follicle development. This allows for greater LH stimulation of follicle growth after deviation than occurs for follicles in the second wave of the ovarian cycle. Dominant follicles that are not maintained by regular (2 hourly) LH pulses will become atretic and fail to ovulate. This is typically the fate of the first wave in the cycle and for the second wave in three-wave cows.

Ovulation is initiated following the large LH spike release that occurs towards the end of the follicular growth curve. The LH surge typically lasts between 5 and 7 hours.

### Oestradiol

Most oestradiol is released by the post-deviation follicles prior to luteinization. Small follicles produce less estradiol than larger follicles and only follicles that develop past deviation produce significant amounts of estradiol. Oestradiol provides negative feedback on FSH release, thus regulating the recruitment of new follicular waves. In pre-pubertal animals the negative feedback loop is highly sensitised resulting in a marked reduction in FSH and GnRH release. This results in failure of recruited follicles to maintain development (therefore leading to follicle regression) and an inability of post-deviation follicles to grow to ovulatory size. This mechanism is thought to be reactivated during periods of stress resulting in anoestrous (e.g. nutritional anoestrous), following parturition (post-partum anoestrous) and in certain breeds whilst lactating (lactational anoestrous).

Cows receiving inadequate nutrition have been demonstrated to have a reduced amplitude and frequency of LH pulses compared to cows on adequate nutrition. This is identical to the LH release pattern seen when the hypothalamus-pituitary axis is over sensitised to the negative feedback effects of oestradiol on FSH and GnRH release. The mechanism linking nutrition to fertility may be mediated via Insulin-Like Growth Factor (ILGF). This again raises a further obstacle to perfectly synchronising the ovulation of cows using a generic programme; the optimal programme varies with individual properties and with season.

Oestrogen exposure will cause regression of FSH dependent (immature) follicles. This effect is indirect and follows the reduction in FSH release following exposure of the hypothalamus-pituitary axis to oestradiol. Removal of ongoing follicle tropism results in death of all follicles within the wave and the next wave begins once the levels of circulating estradiol are reduced. The timing of restoration of FSH levels depends upon the dose and type of oestradiol ester used. Oestrogen exposure does not impact upon post-deviation follicles—these depend upon LH for tropism, but the next follicular wave is suppressed whilst it is present in the system. Care must be taken to ensure that, not only is the effect of the hormone that is injected is considered but the effects of the hormones on which it is dependant must also be taken into account when designing any hormone intervention programme.

### Progesterone

Priming with progesterone before the initiation of a follicular wave results in the development of larger follicles. The mechanism seems to be from the inhibition of LH release when progesterone levels are high: ongoing suppression of LH release may result in an increase in pituitary stores of LH. This may be the reason why cows treated with a priming dose of progesterone experience greater LH pulse amplitude and frequency upon removal of progesterone compared to cows who received no progesterone priming. Larger ovulatory follicles will develop under the improved stimulus arising from the greater amplitude and frequency of LH pulse releases. This effect was shown to carry forward into the life of the CL. Progesterone primed cows were demonstrated to have consistently higher progesterone levels 14 days after AI than cows not primed with progesterone. This may be due to the presence of a larger CL formed from a larger ovulatory follicle. A study demonstrated that doubling of circulating progesterone levels resulted in halving of the frequency of LH pulses. It is clear that any interference with the cow reproductive system has widespread consequences which cannot always be predicted or anticipated.

Progesterone exposure is also thought to ‘reset’ sensitivity of the hypothalamus-pituitary axis to oestradiol and inhibin feedback towards more fertile levels. This mechanism may be the primary mode of action of progesterone exposure in the restoration of cycling activity in anoestrous cows. Anoestrous cows typically have oversensitive negative feedback mechanisms that result in regression of recruited follicles within follicular waves; the dominant follicle is unable to mature sufficiently to attain ovulation size. The system is clearly delicately balanced and any interference by hormone intervention programmes is certain to have widespread consequences.

Progesterone-impregnated inserts can result in the production of an aged oocyte, especially if inserted late into the cycle (when the CL has spontaneously regressed). The ongoing elevation of progesterone levels result in intermediate inhibition of LH release: the follicle grows for longer before the ovulatory LH spike can be initiated. This results in an old and less fertile oocyte being ovulated. Oestradiol release from the developing (aged) follicle over this time may also alter the oviduct environment further inhibiting fertilisation. Timing of any hormone intervention with respect to the cycle phase of the cow will differ between animals again reducing efficiencies of hormone intervention programmes.

The amount of progesterone required for adequate priming may be small. A study that examined the effect of progesterone priming in beef cows (via a used CIDR implant) demonstrated that progesterone priming was effective. This study measured average serum progesterone levels of 1.5 ng/mL in treated (primed) cows compared to levels of 0.4 ng/mL in untreated controls. Hypothalamic progesterone priming systems appear to be more effective when used in conjunction with follicular wave control systems (using GnRH) in improving conception and fertility than either treatment alone. This work has been further defined recently. Only 3-days of elevated progesterone was required to produce significantly a greater maximum and area under the curve for the endogenous LH surge following exposure to progesterone compared to no exposure. Three days of progesterone treatment resulted in significantly larger follicles than untreated animals (13.7 mm compared to 10.5 mm). Progesterone levels of 1-3 ng/mL serum appear to provide effective biological activity.

Progesterone priming is also essential to prevent the uterus from prematurely releasing prostaglandin. This results in a reduction in the incidence of early embryonic loss thereby improving pregnancy rates. The incorporation of progesterone in a hormone intervention programme appears to be very attractive but must be very carefully planned with respect to timing and dose in particular.

### Impact of Production and Nutrition

### Insulin Like Growth Factor

Insulin Like Growth Factor (ILGF) levels within the body are positively correlated to energy and protein levels with energy levels of the body providing the greatest influence upon ILGF levels. Cows in negative energy balance demonstrate a marked decline in ILGF levels. This is by a reduction in ILGF production and also by an increase in the level of bound ILGF (bound ILGF is unavailable). Cows in negative energy balance increase the expression of ILGF binding proteins (ILGFBP), thereby reducing the amount of free (active) hormone. High producing dairy cows in prolonged negative energy balance have low levels of ILGF present after calving.

ILGF has direct activity at the ovarian level. An increase in ILGF activity is required for follicles to become recruited. This occurs by a reduction in the amount of ILGFBP which typically occurs around the time of deviation. The increase in active levels of ILGF enhances follicular responsiveness to LH. FSH also represses the gene coding for ILGFBP production. Therefore the level of circulating ILGF levels determine in part the number of follicles recruited in each wave. Increased levels of ILGF also promote FSH and LH production and activity—some of this effect is to ensure follicles develop effect steroid hormone functionality.

ILGF may be the mechanism for stimulating production of LH receptors on the surface of dominant follicles as cows with low ILGF levels fail to develop large follicles. ILGF may promote angiogenesis within the follicular stroma, therefore promoting an increased blood supply resulting in increased access to LH and follicular growth.

ILGF may provide the association between nutrition, production and reproduction. The increased level of silent oestrus, failed ovulations and early embryonic loss and delayed return to service observed in high production cows may be mediated by a reduction in the level of circulating ILGF. The increased levels and depth of negative energy balance in modern dairy cows appears to be changing the proportion of 2-wave and 3-wave ovarian follicular cycles within the population. The frequency of the less fertile 2-wave cycles appears to be increasing. The mechanism of action is most likely to be a reduction in LH, FSH production and activity arising from increased negative energy balance, with the effect mediated via ILGF.

It is very clear that the nutritional status of the herd and the individuals within the herd of cows undergoing any hormone treatment must be considered carefully in order to obtain maximum and optimal results from the hormone treatments.

### OVsynch

The OVsynch programme has been developed to control oestrus in cows. The programme involves injecting the cows with GnRH on day 1, prostaglandin on day 7 and GnRH on day 9 followed by artificial insemination on day 10. OVsynch has been introduced into several countries throughout the world and is very widely accepted.

The OVsynch programme operates as follows. Follicles between day 4 and day 8 of the follicular wave cycle are most receptive to LH. Therefore, cows that are in dioestrous ovulate more reliably to the first GnRH injection. These cows are between days 5-12 of the ovarian cycle and therefore contain follicle waves that are between 3-9 days old. These follicles have developed LH responsiveness and therefore are most likely to ovulate following exogenous GnRH administration. Pre-deviation follicles are not reliably ovulated and thus not all animals can be reliably synchronised with a single GnRH injection. Studies have demonstrated that conception rates are higher for cows recruited to the programme days 5-12 of the ovarian cycle compared to cows recruited outside these times.

Oestrus display is initiated by the presence of elevated and rising oestradiol levels concurrent with low and falling progesterone levels. This combination occurs around the time of the death of the CL and the maturation of the ovulatory follicle. If the ovulatory follicle is small, then oestradiol production can be inadequate to promote oestrus display. OVsynch cows usually do not display a strong oestrus. This is most likely due to inadequate oestradiol levels arising from the presence of small ovulatory follicles. A higher than normal percentage of cows that fail to become pregnant to fixed time insemination return to oestrus within 18 days when submitted to the OVsynch programme. This may be due to early regression of the CL in some cows. Early regression may be due to failure to produce a functional CL of adequate size arising in turn from the ovulation of a small follicle. Inadequate oestradiol levels may contribute to poor oestrus display observed in cows submitted to the OVsynch programme. Following GnRH2 in the OVsynch programme—GnRH-induced CLs are stimulated that produce less progesterone and a slower rise in P4 levels than spontaneous ovulations.

Exogenous GnRH-induced ovulations tend to result in the formation of CLs that produce less progesterone and have a slower rise in P4 levels than spontaneous ovulations. This may be due to the combined effects of inadequate LH stimulation of follicular development with inadequate oestradiol production by the forming follicle. Oestradiol priming is required by the pituitary to maximise the effectiveness of GnRH at stimulating pituitary release of LH. The development of larger follicles prior to ovulation is likely to result in the formation of more viable and effective CL after ovulation.

Studies have demonstrated that whilst 50 μg of GnRH produced an identical ovulation rate to 100 μg, a dose of 25 μg resulted in dramatic reduction in ovulation rates. However, the LH spike induced by exogenous GnRH is of slightly shorter duration and of lesser magnitude than the LH spike induced as a consequence of endogenous GnRH release. This has been theorised as a possible consequence of producing reduced stimulation for the luteinisation of follicle granulosa cells thereby resulting in formation of a smaller and less active CL. Lower progesterone production by the CL may not provide adequate pregnancy support resulting in an increased loss of conceptus's. These results therefore do not support the use of a dose of GnRH lower than 100 μg within the OVsynch programme.

Progesterone priming between the first GnRH injection and the prostaglandin injection has been shown to result in the development of larger follicles. This is thought to occur because progesterone provides no feedback control over FSH release, thus follicular waves continue to occur despite the presence of progesterone. Progesterone does prevent premature ovulation of the follicle by inhibiting premature LH release. Removal of the progesterone source is essential to allow LH levels to rise and thus initiate ovulation. It has previously been demonstrated that progesterone primed cows had round ovulatory follicles with a 10% larger diameter (1.5 mm) than non-progesterone primed cows.

Progesterone priming of 3-4 days may be sufficient to reset the hypothalamus-pituitary sex hormone feedback sensitivity mechanisms. It may also promote the build up of natural LH stores in the pituitary. This may provide for greater frequency and amplitude of LH releases from the pituitary after deviation has occurred resulting in the initiation of ovulation of a larger follicle by the second GnRH injection within the programme. All exogenous progesterone must be removed before the prostaglandin injection because residual progesterone will result in inhibition of LH release, thereby reducing growth of the dominant follicle. Administration of GnRH has been demonstrated to produce a refractory effect on subsequent LH release from the pituitary. Therefore, a suboptimal dose of GnRH administered with the prostaglandin injection must be followed up by a complete dose (100 μg) of GnRH two days later to ensure enough LH is released to promote ovulation.

Cows that received a progestogen treatment administered between the first GnRH injection and the prostaglandin injection had a higher conception rate than cows that did not receive the progestogen treatment. This treatment was demonstrated to result in fewer cows on heat before completing the course and a reduction in the number of cows not pregnant to artificial insemination that returned to heat within 18 days of insemination.

### OVsynch Modifications

### Presynch

### PG(1)-PG(14)-GnRH(d26)-PG(d33)-GnRH(d35)-AI(d36)

The Presynch programme was developed for cycling cows. This system uses a 14-day double prostaglandin (14-DPG) treatment programme initiated 24-26 days before an OVsynch programme. The 14-DPG programme resets the luteal phase and this promotes the ovulation of the dominant follicle of the wave occurring at the death of the CL. The next follicular wave is initiated between 1 and 5 days after the final injection of the 14-DPG. Therefore, a follicle that is responsive to LH (or is already regressing) is present by the time the OVsynch programme is due to start. This system is not effective for anoestrous cows as they do not possess a CL. The programme also takes 34-36 days from initial recruitment to AI. This is not practicable in seasonal calving systems as a large proportion of cows will be calved less than 40 days (and are therefore anoestrous) when the Presynch programme needs to be started for a FTAI to occur on the first day of the mating programme. Presynch systems may be suitable for batch-mating systems that involve voluntary waiting periods for most cows (thereby giving them a chance to begin cycling activity). The Presynch-treated cycling cows have been shown to have around a 10% higher pregnancy rate than OVsynch-treated cycling cows in US studies. At present it appears that it may be impractical and has therefore not received widespread acceptance.

### Heatsynch

GnRH(d1)-PG(d7)-ECP(d8)-AI (d10) (ECP=1-2 mg Estradiol Cypionate)

The Heatsynch system is a modification of the basic OVsynch programme that replaces the final GnRH injection on day 9 with an injection of estradiol cypionate (ECP) given instead on day 8 of the programme. ECP is used to replace GnRH because ECP will induce a LH surge to occur. This LH surge is around 40 hours after administration of ECP; therefore it must be given no later than day 8 of the programme. ECP will also promote the display of oestrus. This characteristic along with the varying timing of the LH surge requires the detection of heat—FTAI systems are not suited to this programme. In general, Heatsynch results in higher oestrus rates, reduced LH surges, reduced ovulation rates, and a slightly reduced pregnancy rate when compared to OVsynch.

A Heatsynch study that examined the combined use of visual heat detection and FTAI for all cows not detected on heat to FTAI at between 65-74 hours after the prostaglandin injection demonstrated no advantage over a simple Heatsynch system that did not blanket mate all cows not observed on heat. Heatsynch was developed to reduce the cost of mating programmes; however the cost of GnRH has reduced greatly since development and ECP is no longer registered for use in many countries. It appears to show no economic advantages to the producer and has therefore not received market acceptance.

### Cosynch

### GnRH(d1)-PG(d7)-GnRH and AI(d9) at 48 to 60 Hours After PG

The Cosynch system was developed for beef production. This modification of the basic OVsynch system delays the second GnRH injection to between 48-60 hours after the prostaglandin injection (which is set at 48 hours after prostaglandin in the OVsycnh programme). Cows are submitted for FTAI at the time of the second GnRH injection. This system results in once less handling period for the stock and hastens the insemination of cows. Studies comparing Cosynch to OVsynch in dairy cows found Cosynch was associated with a slight reduction in pregnancy rates compared to OVsynch whilst comparable results have been demonstrated when this system is used in beef cows. The reduced handling appears to have no advantage to dairy cows and the lack of economic benefit that has been demonstrated has meant that the programme has not been accepted in commercial enterprises.

### Resynch

GnRH(d1)-PG(d7)-GnRH(d9)-AI(d10)-GnRH(d21)-PregTest(D28+PG(if empty)-GnRH (D30 empty cows)-FTAI(d31 empty cows)

The Resynch system is a development designed to hasten re-insemination of cows not pregnant to FTAI. This programme was developed for large herds that use batch mating systems. Cows that were previously submitted to an OVsynch programme are injected with GnRH 21-23 days after FTAI and 7 days before pregnancy testing with ultrasound (at 28-30 days after FTAI). Cows not detectably pregnant receive a prostaglandin injection at the time of pregnancy test, GnRH injection 2 days later (30-32 days after FTAI) and a repeat FTAI the next day (31-33 days after first FTAI). This system increases the pregnancy rate within a set mating period by maximising the number of opportunities to mate empty cows. However, studies have demonstrated that early initiation of Resynch can result in reduced fertility at that mating. This study also demonstrated an adverse interaction between use of Bovine Somatotropin (bST) and GnRH administration in pregnant cows which has also been observed to manifest itself as an increased pregnancy loss. This system would require manual pregnancy testing (without ultrasound) to be delayed to 42 days after FTAI to allow for accuracy of diagnosis and to time GnRH injections with the follicular waves of cows. This appears not to be feasible in most dairies and therefore the system has not been widely utilised.

### Modified Targeted Breeding Programme

PG(d1)-GnRH(d14)-PG(d7)-heat detect and AI from d7-d9 FTAI all remaining D10

The Modified Targeted Breeding (MTB) programme uses prostaglandin and GnRH to hasten the return to service of cows detected to not be pregnant. Prostaglandin is administered to cows detected to be not pregnant and this is followed in 14 days with an injection of GnRH. A second prostaglandin injection is administered seven days after the GnRH injection and cows are submitted for service on detected heat. Any cow that has not been mated by 10 days after the second prostaglandin injection is submitted to FTAI. MTB uses GnRH to ensure ovulation of the dominant follicle thus initiating development of the CL. This CL is receptive to prostaglandin seven days later. Therefore MTB systems synchronise both the luteal and follicular cycle and provide the option to mate cows to detected heat. The system is cumbersome and the improvements are marginal so has only been accepted in a small proportion of dairies.

In conclusion, it appears that the OVsynch protocol is a highly effective reproductive management tool. OVsynch provides significant advantages over other programmes including a is simple injection-based drug delivery system, no requirement for heat detection and efficacy in both cycling and anoestrous cows. The programme can be applied in seasonal calving, year-round and batch mating systems. Intensive research of the OVsynch protocol is currently being undertaken and further advancements in ease of use and performance can be expected.

### Example 1

### Improvements to the OVsynch Protocol

### 1. Introduction

The ‘OVsynch’, or GPG, oestrus synchronization protocol uses a combination of GnRH and prostaglandin, followed by fixed time insemination (FTAI). The usefulness of the protocol in whole herd synchrony programmes has been demonstrated in studies around the world and in a local field study conducted by Parnell Laboratories in conjunction with dairy veterinarians in Victoria in 2001. The application of OVsynch to the treatment and synchronization of anovulatory anoestrus (M) cows is supported by a small body of published work and the results of a major study in pasture-based cows in Australia and New Zealand conducted by Parnell Laboratories.

There is much discussion in the literature as to how to increase the number of cows that become pregnant as a consequence of the oestrus synchronization programmes.

A preliminary study was conducted in order to assess the possible synergistic effect of incorporation of a progesterone treatment into the GPG protocol. It has been hypothesised that a major cause of failure of ovulation in AA cows is due to a lack of LH (frequency and amplitude) arising predominantly from an accentuation of the negative feedback loop of oestradiol on the Pituitary. This results in a failure of sufficient LH release to allow follicle development past deviation, maturation and ovulation and the failure of a sufficiently large and active Corpus Luteum (CL) to develop. It is thought that the provision of progesterone for a sustained period (with its negative feedback effects upon GnRH release) will result in damping down of pituitary activity with a ‘resetting’ of the pituitary feedback loop sensitivity to normal levels. When activity is restored, the negative effect of oestradiol on the pituitary is lessened thus allowing release of LH from the pituitary and this stimulates maturation and ovulation of follicles beyond deviation. This may possibly be the mechanism of action of progesterone releasing devices in the treatment of AA cows.

The incorporation of 4-5 days of progesterone into the first GnRH injection of the GPG protocol was examined as a means of supporting ovulation of a mature follicle, and the development of a healthy and active CL.

### 2. Objective

The objective of the study was to compare the efficacy of the standard GPG protocol with a GPG-P protocol (incorporating 250 mg of progesterone with the first GnRH injection) as a treatment for AA cows. The comparison of efficacy was via measurement of the conception rate to fixed time insemination (FTAI), 3- and 6-week pregnancy rates, and the distribution patterns of returns to service. This study used three herds and around 400 AA cows in order to provide a guide to effectiveness.

### 3. Study Design

Approximately 400 anovulatory anoestrous cows (subject to inclusion criteria) were identified before mating in a number of herds in Australia. Cows were subsequently submitted to either the GPG-P or GPG treatment protocols followed by FTAI. Heat detection aids (KAMARS® or Estrus Alerts®) were applied to all cows and heat detection was performed for at least 25 days after FTAI and all heats were recorded. All AA cows were pregnancy tested between 12 and 15 weeks after FTAI and stage of pregnancy was determined.

### 4. Data Analysis

The FTAI conception rate, 3-week (21-day) and 6-week (42-day) in-calf rates were determined for all cows in the study herds. Comparison was made to the latest data from the Dairy Research and Development Corporation ‘In-Calf’ Project and previously published data for anovulatory anoestrous cows. Results were compared between cows treated with GPG-P and GPG.

### 5. Power Analysis

A study of 400 anoestrous cows (200 per group) can be expected to provide a 95% confidence interval (ie. 95% Cl 30.3%-44.1%) of around an expected average conception rate of 37.0%.

### 6. Study End-Points

The following indices were generated for each herd, for the anoestrous cows in each herd, and for GPG-P and GPG anoestrous cows in the study overall:
FTAI conception rates
3-week pregnancy rates
6-week pregnancy rates
Return to service distribution after FTAI

The start point for the study, identified as day 0, was FTAI. All time related indices were calculated relative to this date.

### 7. Results

Three farms provided AA cows for inclusion into the study. These are described below:

| | | | | Days from | Pregnancy |
|---|---|---|---|---|---|
| | | | | FTAI to | detectable |
| Farm | MSD | FTAI | PT Date | PT | days |
| Farm 1 | 1/11/2003 | 19/11/2003 | 26/02/2004 | 99 | 64 |
| Farm 2 | 1/11/2003 | 10/12/2003 | 26/02/2004 | 78 | 43 |
| Farm 3 | 1/11/2003 | 17/11/2003 | 3/03/2004 | 107 | 72 |

The distribution of AA and AA trialists (cows providing all data for analysis) is given below:

| | Total herd | Total trialist |
|---|---|---|
| Farm | AA cows | AA cows |
| Farm 3 | 150 | 145 |
| Farm 2 | 150 | 95 |
| Farm 1 | 156 | 123 |

The distribution of trialists by treatment group (C=control and P=Progesterone) is given below.

| | | Number |
|---|---|---|
| Farm | Group | of trialists |
| Farm 3 | C | 93 |
| | P | 52 |
| Farm 2 | C | 54 |
| | P | 41 |
| Farm 1 | C | 80 |
| | P | 43 |

| | Total trialists |
|---|---|
| Group | by group |
| C | 227 |
| P | 136 |

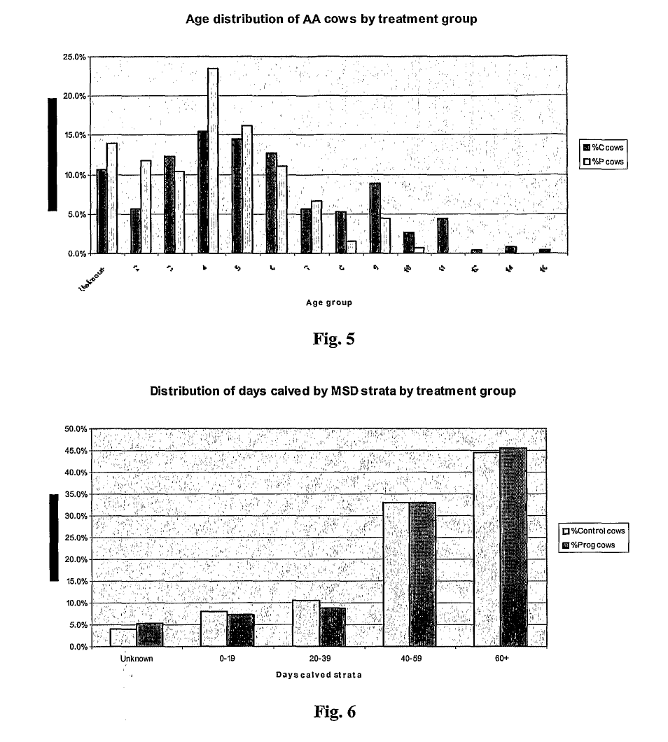

A total of 363 AA cows provided data for analysis. The distribution of cow age by treatment group is given in FIG. 5. The distribution of number of days calved by MSD strata by treatment group is given In FIG. 6.

An experienced ultrasound pregnancy tester was used to undertake all trial pregnancy testing work. An algorithm (as used by InCalf) was employed to assign pregnancies to heat dates. Any heat date within 3 days of the pregnancy testing estimated conception date was assigned, otherwise any heat date occurring up to 14 days after the estimated conception date was assigned otherwise, any heat 14 days prior to the estimated conception date was assigned otherwise a new heat (missed heat) was assigned.

The distribution of results for FTAI are given below:

| | | | | | | | p-value |
|---|---|---|---|---|---|---|---|
| | | | Not | Preg- | % | | (Chi |
| Farm | Group | Total | pregnant | nant | Pregnant | RR | square) |
| Farm 3 | C | 93 | 63 | 30 | 32.3% | — | 0.67 |
| | P | 52 | 37 | 15 | 28.8% | 0.89 | |
| Farm 2 | C | 54 | 41 | 13 | 24.1% | — | 0.12 |
| | P | 41 | 25 | 16 | 39.0% | 1.62 | |
| Farm 1 | C | 80 | 63 | 17 | 21.3% | — | 0.02 |
| | P | 43 | 25 | 18 | 41.9% | 1.97 | |
| Total | C | 227 | 167 | 60 | 26.4% | — | 0.05 |
| | P | 136 | 87 | 49 | 36.0% | 1.36 | |

The distribution of results for 21-day pregnancy are given below:

| | | | | | | | p-value |
|---|---|---|---|---|---|---|---|
| | | | Not | Preg- | % | | (Chi |
| Farm | Group | Total | pregnant | nant | Pregnant | RR | square) |
| Farm 3 | C | 93 | 57 | 36 | 38.7% | — | 0.98 |
| | P | 52 | 32 | 20 | 38.5% | 0.99 | |
| Farm 2 | C | 54 | 38 | 16 | 29.6% | — | 0.06 |
| | P | 41 | 21 | 20 | 48.8% | 1.65 | |
| Farm 1 | C | 80 | 47 | 33 | 41.3% | — | 0.19 |
| | P | 43 | 20 | 23 | 53.5% | 1.30 | |
| Total | C | 227 | 142 | 85 | 37.4% | — | 0.10 |
| | P | 136 | 73 | 63 | 46.3% | 1.24 | |

The distribution of results for 42-day pregnancy are given below:

| | | | | | | | p-value |
|---|---|---|---|---|---|---|---|
| | | | Not | Preg- | % | | (Chi |
| Farm | Group | Total | pregnant | nant | Pregnant | RR | square) |
| Farm 3 | C | 93 | 48 | 45 | 48.4% | | 0.35 |
| | P | 52 | 31 | 21 | 40.4% | 0.83 | |
| Farm 2 | C | 54 | 30 | 24 | 44.4% | | 0.26 |
| | P | 41 | 18 | 23 | 56.1% | 1.26 | |
| Farm 1 | C | 80 | 38 | 42 | 52.5% | | 0.27 |
| | P | 43 | 16 | 27 | 62.8% | 1.20 | |
| Total | C | 227 | 116 | 111 | 48.9% | | 0.54 |
| | P | 136 | 65 | 71 | 52.2% | 1.07 | |

The distribution of results for total pregnancy are given below:

| | | | | | | | p-value |
|---|---|---|---|---|---|---|---|
| | | | Not | Preg- | % | | (Chi |
| Farm | Group | Total | pregnant | nant | Pregnant | RR | square) |
| Farm 3 | C | 93 | 33 | 60 | 64.5% | — | 0.56 |
| | P | 52 | 21 | 31 | 59.6% | 0.92 | |
| Farm 2 | C | 54 | 30 | 24 | 44.4% | — | 0.37 |
| | P | 41 | 19 | 22 | 53.7% | 1.21 | |
| Farm 1 | C | 80 | 30 | 50 | 62.5% | — | 0.59 |
| | P | 43 | 14 | 29 | 67.4% | 1.08 | |
| Total | C | 227 | 93 | 134 | 59.0% | — | 0.81 |
| | P | 136 | 54 | 82 | 60.3% | 1.02 | |

The distribution of results by cow age are given below:

| | | | | | | | p-value |
|---|---|---|---|---|---|---|---|
| | | | Not | Preg- | % | | (Chi |
| Age | Group | Total | pregnant | nant | Pregnant | RR | square) |
| Un- | C | 24 | 19 | 5 | 20.8% | | 0.42 |
| known | P | 19 | 13 | 6 | 31.6% | 1.52 | |
| 2 | C | 13 | 9 | 4 | 30.8% | | 0.98 |
| | P | 16 | 11 | 5 | 31.3% | 1.02 | |
| 3 | C | 28 | 18 | 10 | 35.7% | | 1.00 |
| | P | 14 | 9 | 5 | 35.7% | 1.00 | |
| 4-7 | C | 110 | 80 | 30 | 27.3% | | 0.05 |
| | P | 78 | 46 | 32 | 41.0% | 1.50 | |
| 8+ | C | 52 | 41 | 11 | 21.2% | | 0.48 |
| | P | 9 | 8 | 1 | 11.1% | 0.53 | |

The distribution of results by number of days calved by MSD strata are given below:

| Days | | | Not | | | | p-value |
|---|---|---|---|---|---|---|---|
| Calved Strata | Group | Total | pregnant | Pregnant | % Pregnant | RR | (Chi square) |
| Unknown | C | 9 | 7 | 2 | 22.2% | | 0.69 |
| | P | 7 | 6 | 1 | 14.3% | 0.64 | |
| 0-19 | C | 18 | 17 | 1 | 5.6% | | 0.24 |
| | P | 10 | 8 | 2 | 20.0% | 3.60 | |
| 20-39 | C | 24 | 14 | 10 | 41.7% | | 1.00 |
| | P | 12 | 7 | 5 | 41.7% | 1.00 | |
| 40-59 | C | 75 | 53 | 22 | 29.3% | | 0.65 |
| | P | 45 | 30 | 15 | 33.3% | 1.14 | |
| 60+ | C | 101 | 76 | 25 | 24.8% | | 0.02 |
| | P | 62 | 36 | 26 | 41.9% | 1.69 | |
| Short calved | C | 117 | 84 | 33 | 28.2% | | 0.51 |
| (<60 days) | P | 67 | 45 | 22 | 32.8% | 1.16 | |
| Long calved | C | 101 | 76 | 25 | 24.8% | | 0.02 |
| (>60 days) | P | 62 | 36 | 26 | 41.9% | 1.69 | |

The percentage of cows not pregnant to FTAI that returned to subsequent heat is given below:

No return

| | |
|---|---|
| C | 21.6% |
| P | 29.9% |

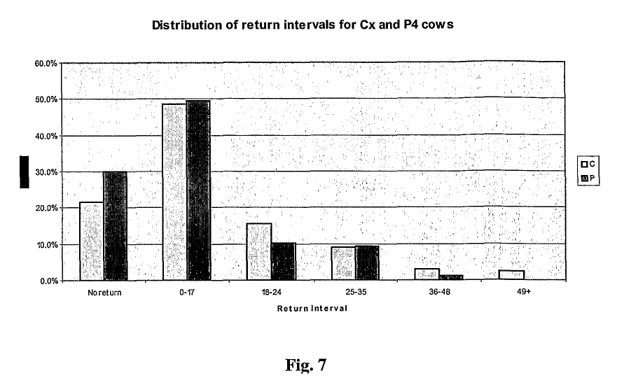

The distribution of return intervals for cows not pregnant to FTAI is given in FIG. 7.

### Conclusions:

The conclusions that can be drawn from this study investigating the role of injectable progesterone within a standard OVsynch protocol are:
1. Ov-P4 significantly improves fertility to the FTAI round. The difference in FTAI conception rate in AA cows between control and P4 cows was found to be around 10% (26.4% compared to 36%). This difference was significant (p=0.05) even in this small powered study.
2. The improvement in FTAI performance in AA cows seems to be due to a reduction in inter-farm variation in FTAI conception rates. The range of FTAI performances on all farms were 21.3-32.3% for C and 28.3-41.9% for P4 treated cows. The difference between treatment groups within farms was −3.4%, 15.0% and 9.6% for P4 FTAI CR compared to controls.
3. The strata of cows that appeared to respond the most favourably to P4 supplementation of the OVsynch protocol were AA cows that were long calved by MSD. This supports the observation that progesterone priming may assist by ‘resetting’ pituitary-hypothalamic sensitivity to feedback loops of sex hormones. Long calved AA cows have no real reason not to resume cycling. The erroneous functioning of the pituitary loop may explain why this group of cows has a delayed return to oestrus. The ‘resetting’ effect of the P4 injection may explain the difference between the two groups in this study
4. There was no obvious age effect on treatment.
5. Ov-P4 does not seem to enhance fertility in M cows not conceiving to FTAI by reducing the incidence of Phantom cows
6. Ov-P4 does not alter the pattern of returns to oestrus in cows not pregnant to FTAI
7. Ov-P4 does not inhibit subsequent fertility in treated AA cows

### Example 2

### Formulation of Product

In order to manufacture the product, it would be ideal if the GnRH and the progesterone were in the same injection. In order to achieve this, it is necessary to devise means by which to formulate two very different molecules into a stable formulation. GnRH is a decapeptide, i.e. a short protein containing ten amino acids. This material is particularly soluble in aqueous solutions. Progesterone on the other hand is a hydrophobic molecule that is soluble in non-aqueous solvents but not soluble in aqueous solvents.

A further requirement of the final formulation is that it be possible to sterilise the material by filtration through 0.2 μm filters. Neither the progesterone nor the GnRH can be sterilised by autoclaving as they are unstable to heat. In order to achieve a stable formulation, a large number of variables have to be tested.

The following examples illustrate the types of formulation that may be suitable.

### Formulation 1

| Material | mg |
|---|---|
| Progesterone BP/USP | 250 |
| GnRH Acetate | 0.1 |
| Plasdone C15 | 50 |
| Water | 10 |
| N-methyl-2-pyrrolidone | to 2.0 mL |

The formulation protocol involves dissolving the GnRH in the water in one vessel. The progesterone is dissolved in a portion of the N-methyl-2-pyrrolidone, the Plasdone C15 is added and dissolved, the GnRH/water is then added and the mixture diluted to volume with N-methyl-2-pyrrolidone. This has been found to form a stable solution upon storage at ambient and elevated temperatures.

This formulation has no preservative. However since there is a very low level of water in the mixture, it is considered unlikely that it will support the growth of microorganisms. However, for some applications, it may be decided advantageous to add a preservative. Benzyl alcohol has been found to be compatible with the formulation under development. This can be formulated as follows.

### Formulation 2

| Material | mg |
|---|---|
| Progesterone BP/USP | 250 |
| GnRH Acetate | 0.1 |
| Benzyl Alcohol | 20 |
| Plasdone C15 | 50 |
| Water | 10 |
| N-methyl-2-pyrrolidone | to 2.0 mL |

The formulation protocol involves dissolving the GnRH in the water in one vessel. The progesterone is dissolved in a portion of the N-methyl-2-pyrrolidone, the Plasdone C15 and benzyl alcohol are added and dissolved, the GnRH/water is then added and the mixture diluted to volume with N-methyl-2-pyrrolidone. This has been found to form a stable solution upon storage at ambient and elevated temperatures.

### Formulation 3

| Material | mg |
|---|---|
| Progesterone BP/USP | 250 |
| GnRH Acetate | 0.1 |
| Plasdone C15 | 50 |
| Water | 10 |
| Glycerol formal | to 2.0 mL |

The formulation protocol involves dissolving the GnRH in the water in one vessel. The progesterone is dissolved in a portion of the glycerol formal, the Plasdone C15 is added and dissolved, the GnRH/water is then added and the mixture diluted to volume with glycerol formal. This has been found to form a stable solution upon storage at ambient and elevated temperatures.

This formulation has no preservative. However since there is a very low level of water in the mixture, it is considered unlikely that it will support the growth of microorganisms. However, for some applications, it may be decided advantageous to add a preservative. Benzyl alcohol has been found to be compatible with the formulation under development. This can be formulated as follows.

### Formulation 4

| Material | mg |
|---|---|
| Progesterone BP/USP | 250 |
| GnRH Acetate | 0.1 |
| Benzyl Alcohol | 20 |
| Plasdone C15 | 50 |
| Water | 10 |
| Glycerol formal | to 2.0 mL |

The formulation protocol involves dissolving the GnRH in the water in one vessel. The progesterone is dissolved in a portion of the Glycerol formal, the Plasdone C15 and benzyl alcohol are added and dissolved, the GnRH/water is then added and the mixture diluted to volume with N-methyl-2-pyrrolidone. This has been found to form a stable solution upon storage at ambient and elevated temperatures.

### Example 3

### Further Formulations

### Process for all Formulations:

1) The Gonadorelin acetate was dissolved in the water.
2) The first quantity of N-methyl-2-pyrrolidone was added to the solution obtained in step 1.
3) The progesterone, benzyl alcohol and Plasdone C15 were dissolved in the solution obtained in step 2.
4) Additional N-methyl-2-pyrrolidone was added was added to volume.

| Material | mg | (Kg) |
|---|---|---|
| Progesterone BP/USP | 250 | 125 |
| Gonadorelin Acetate | 0.1 | 0.05 |
| Benzyl Alcohol | 20 | 10 |
| Plasdone C15 | 50 | 25 |
| water | 50 | 25 |
| N-methyl-2-pyrrolidone | 1727 | 863.5 |
| N-methyl-2-pyrrolidone | To 2.0 mL | to 100 L |
| Progesterone BP/USP | 250 | 125 |
| Gonadorelin Acetate | 0.1 | 0.05 |
| Benzyl Alcohol | 20 | 10 |
| Plasdone C15 | 50 | 25 |
| water | 25 | 12.5 |
| N-methyl-2-pyrrolidone | 1752 | 876 |
| N-methyl-2-pyrrolidone | To 2.0 mL | to 100 L |
| Progesterone BP/USP | 250 | 125 |
| Gonadorelin Acetate | 0.1 | 0.05 |
| Benzyl Alcohol | 20 | 10 |
| Plasdone C15 | 50 | 25 |
| water | 10 | 5 |
| N-methyl-2-pyrrolidone | 1766 | 883 |
| N-methyl-2-pyrrolidone | To 2.0 mL | to 100 L |

### Example 4

The purpose of the formulation studies was to establish a product vehicle that was suitable for solubilisation of a relatively high concentration of Progesterone but in which Gonadorelin would be stable. Literature studies indicated that most of Progesterone products were in the form of a suspension, which was not desirable from a marketing perspective. Low solubility of Progesterone (even in organic solvents) further limited the choice of injectable organic vehicles.

### Progesterone Solubility Trials:

Solubility of Progesterone was found to be marginal in the following pure solvents:

### PEG400

### Propylene Glycol

### Tetraglycol

Further Solubility Trials were conducted as follows:

### Solubility Trial 1:

### Progesterone BP, 0.25 g

### 2-Pyrrolidone, 0.32 g

### Benzyl Alcohol, 0.02 g

### Propylene Glycol, to 1 mL

This trial resulted in a white slurry emulsion.

### Solubility Trial 2:

### Progesterone BP, 0.25 g

### Ethanol, 0.40 g

### Benzyl Alcohol, 0.20 g

### 2-Pyrrolidone, to 1 mL

This trial resulted in a slightly white slurry emulsion.

### Solubility Trial 3:

### Progesterone BP, 0.25 g

### Ethanol, 0.04 g

### Benzyl Alcohol, 0.02 g

### 2-Pyrrolidone, to 2 mL

This trial resulted in a very slightly white slurry emulsion.

### Solubility Trial 4:

### Progesterone BP, 0.25 g

### Benzyl Alcohol, 0.02 g

### N-Methyl 2-Pyrrolidone, to 1 mL

This trial resulted in a very slightly white slurry emulsion.

### Solubility Trial 5:

### Progesterone BP, 0.25 g

### Benzyl Alcohol, 0.02 g

### N-Methyl 2-Pyrrolidone, to 2 mL

This trial resulted in a clear and colourless solution.

### Solubility Trial 6:

### Progesterone BP, 0.25 g

### Benzyl Alcohol, 0.02 g

### N-Methyl 2-Pyrrolidone, to 1.5 mL

This trial resulted in a clear and colourless solution. After 6 days at room temperature precipitation was observed in Solution 6 with N-Methyl 2-Pyrrolidine (NMP), hence Solution 5 was further investigated in further scale up and solubility trials.

This preliminary work indicated that NMP was the best solvent for dissolution of Progesterone. This was further confirmed by solubility testing performed at later development stages.

### Gonadorelin Solubility Trials:

### Solubility Trial 7:

### Progesterone BP, 250 mg

### Gonadorelin Acetate, 0.1 mg

### Benzyl Alcohol, 20 mg

### PVP K30, 50 mg

### NMP, to 2 mL

Gonadorelin did not dissolve; therefore attempts were made to dissolve Gonadorelin Acetate in water, followed by addition of other ingredients

### Solubility Trial 8:

### Gonadorelin Acetate, 0.0013 g

### Water, 0.1 g

### Progesterone BP, 1.25 g

### Benzyl Alcohol, 0.1 g

### PVP K30, 0.26 g

### NMP, to 10 mL

This trial resulted in clear solution that did not precipitate. The colour of the solution yellowed slightly over time, which was attributed to the Polyvinyl Povidone PVP K30.
Preservative Efficacy Test (BP) was performed with Solubility Trial 8 as the base formulation. Solutions were prepared both with and without benzyl alcohol. Both solutions passed the preservative efficacy test therefore it was decided to omit benzyl alcohol from the formulation.

### Solubility Trial 9:

Further solubility trials were performed to investigate the potential of Glycerol Formal as vehicle.

### Gonadorelin Acetate, 0.005 g

### Water, 0.5 g

### Progesterone BP, 12.5 g

### Glycerol Formal, to 100 mL

As it took 40 minutes to dissolve Progesterone in the glycerol formal, NMP remained the primary vehicle candidate.
To increase the water content in order to improve Gonadorelin solubility, the following samples were prepared:

| Ingredient | RD0306/2 | RD0306/3 |
|---|---|---|
| Gonadorelin Acetate | 0.001 g | 0.001 g |
| Water | 0.5 g | 1.0 g |
| Progesterone | 2.5 g | 2.5 g |
| Plasdone C15 | 0.5 g | 0.5 g |
| NMP | To 20 mL | To 20 mL |
| Observation | Progesterone | Progesterone did |
| | fully dissolved | not fully dissolve |

This led to the conclusion that the maximum water level for dissolution of Gonadorelin without affecting solubility of Progesterone was between 2.5% and 5%.

A detailed investigation of the maximum water level was conducted as detailed below:

| Ingredient | RD0306/4 | RD0306/5 | RD0306/6 | RD0306/7 |
|---|---|---|---|---|
| Gonadorelin | 0.0025 g | 0.0025 g | 0.0025 g | 0.0025 g |
| Acetate | | | | |
| Water | 1.50 g | 1.75 g | 2.0 g | 1.25 g |
| Progesterone | 6.25 g | 6.25 g | 6.25 g | 6.25 g |
| Plasdone C15 | 1.25 g | 1.25 g | 1.25 g | 1.25 g |
| NMP | To 50 mL | To 50 mL | To 50 mL | To 50 mL |

| | |
|---|---|
| Observation | No precipitation observed |

RD0306/4-7 samples were stored at room temperature (wrapped in Aluminium foil) and were re-tested after 39 days, with the following results:

| Ingredient | RD0306/4 | RD0306/5 | RD0306/6 | RD0306/7 |
|---|---|---|---|---|
| Gonadorelin Acetate (%) | 82.0 | 78.9 | 75.6 | 76.3 |

Progesterone content remained virtually unchanged in the prepared samples. Testing showed that a water content of 3% was the most appropriate for both solubilisation and stability of Gonadorelin. Nevertheless, Gonadorelin degradation after five weeks indicated that the formulation did not demonstrate an acceptable stability profile. A literature study on NMP indicated that the pH of this organic solvent was approximately 11.5, which was incompatible with the protein structure of Gonadorelin. Although not appropriate for accurate pH measurement of organic solvents, a pH meter was used to monitor pH adjustments. Attempts to lower the solution pH by the addition of buffering salts to the solution were unsuccessful due to the low water content. Therefore, reduction of pH to approximately pH=4.5 was achieved by addition of the following acids:
Glacial Acetic Acid—approximately 10% of Acetic Acid was required to lower the pH. This volume would reduce the amount of NMP which could lead to Progesterone precipitation. Consequently, Acetic acid was abandoned in further studies.
Concentrated Phosphoric Acid—after addition of approx. 1% Phosphoric acid, pH was lowered to approximately pH=5; 1.5-2% acid did not reduce pH to less than 4.5, indicating that at that level of NMP/Acid combination the solution reached a buffering equilibrium.
Concentrated Formic Acid and 0.25% (w/v) Hydrochloric Acid—Addition of these acids resulted with approximately the same pH modification as that already achieved with Phosphoric Acid.

Since addition of acids reduced the amount of NMP available for dissolution of Progesterone, samples prepared with different acids were refrigerated at 2-8° C. to investigate progesterone solubility and the occurrence of precipitation at low temperatures. The appearance of crystals in samples prepared with formic and hydrochloric acid was obvious after several hours of refrigeration. Crystals appeared after 1-2 days in samples prepared with phosphoric acid. FTIR testing confirmed that the precipitate was crystalline progesterone, which was difficult to redissolve due to the crystalline form of the precipitate. At this stage a further search for vehicle that would provide sufficient progesterone solubility at low temperatures was conducted. The following vehicles were used:
N,N-Dimethyl Acetamide
N,N-Dimethyl Acetamide with Benzyl Alcohol (as solubilizer)
NMP with 10% Solutol
NMP with 10% Ethanol
NMP with 5% Polyvinyl Povidone K-30 as stabilizer
NMP with Sulfuric Acid for pH adjustment
NMP with Oxalic Acid for pH adjustment
NMP with Methane Sulfonic Acid for pH adjustment
NMP with 10% and 15% Benzyl Alcohol (as solubilizer)
Glycerol Formal with and without Benzyl Alcohol (as solubilizer)
2-Pyrrolidone in combination with Formic or Phosphoric acid
   None of the above combinations resulted in acceptable Progesterone solubility at low temperatures and stable Gonadorelin. A solubility study confirmed that the most appropriate combination was NMP+water+phosphoric acid with progesterone amount not exceeding 8-10% w/v.

Additionally it was during this phase of the development studies that it was determined via literature review regarding progesterone uptake, theorized required blood levels and efficacy that a concentration of progesterone 7.5% was likely to be sufficient to achieve the desired physiological effect.

Therefore the following formulation was investigated:

| | | |
|---|---|---|
| Gonadorelin (as Acetate) | 0.05 | mg/mL |
| Progesterone | 75 | mg/mL |
| Conc. H₃PO₄ (85%) | 15 | mg/mL |
| Water for Injection | 30 | mg/mL |
| N-Methylpyrrolidone | to 1 | mL |

Gonadorelin results for two laboratory batches stored at ambient conditions in the laboratory were as follows:

| Days | RD347 (%) | RD354 (%) |
|---|---|---|
| 0 | 100.0 | 100.0 |
| 5 | n/a | 98.7 |
| 15 | 97.1 | n/a |
| 21 | n/a | 98.5 |
| 32 | n/a | 99.2 |
| 35 | 96.1 | n/a |
| 42 | n/a | 97.4 |
| 49 | 96.4 | n/a |
| 60 | 94.8 | n/a |
| 95 | n/a | 91.6 |
| 115 | 89.5 | n/a |

The above results indicated that the reduction in formulation pH resulted in potential extension of shelf life to approximately 3-4 months, indicating that further development to limit degradation was necessary.

Investigation of suitable antioxidants for inclusion in the formulation was conducted whilst bearing in mind that addition of an antioxidant may reduce Progesterone solubility at low temperature. Hence, any appropriate antioxidant had to be highly effective at very low concentration. A literature search indicated that Butylated Hydroxyanisole (BHA), Butylated Hydroxytoluene (BHT) and Propyl Gallate (PGal) alone or in combination with each other (due to the synergistic effect) may be appropriate.

Generally, antioxidants are light sensitive (sensitivity lowest with BHA and highest with BHT); based on previous experience with a Gonadorelin based product it was known that Gonadorelin is also light sensitive. Samples (tabled below) were therefore exposed to UV light (254 nm) to:
1. Determine the appropriate antioxidant combination, and
2. Determine whether the presence of an antioxidant reduces the rate of Gonadorelin degradation when exposed to light

| | | | RD0354 | RD0366 | RD0367 | RD0369 | RD0370 |
|---|---|---|---|---|---|---|---|
| Gonadorelin (as Acetate) | 0.05 | mg/mL | + | + | + | + | + |
| Progesterone | 75 | mg/mL | + | + | + | + | + |
| Conc. H3PO4 (85%) | 15 | mg/mL | + | + | + | + | + |
| Water for Injection | 30 | mg/mL | + | + | + | + | + |
| BHA | 0.2 | mg/mL | n/a | + | + | n/a | + |
| BHT | 0.2 | mg/mL | n/a | + | n/a | n/a | n/a |
| Propyl Gallate | 0.2 | mg/mL | n/a | n/a | n/a | + | + |
| N-Methylpyrrolidone | to 1 | mL | + | + | + | + | + |

After six days of exposure to the UV light at 254 nm results were as follows:

| | RD0354 | RD0366 | RD0367 | RD0369 | RD0370 |
|---|---|---|---|---|---|
| Days exposed to UV | 4 | 6 | 6 | 6 | 6 |
| light | | | | | |
| Gonadorelin (as Acetate) | 85.6 | 91.1% | 89.5% | 87.7% | 90.9% |
| Approx. Rate of | 3.6% | 1.5% | 1.8% | 2.0% | 1.5% |
| Gonadorelin | | | | | |
| Degradation (%/day) | | | | | |
| BHA | n/a | 95.6% | 76.5% | n/a | 88.0% |
| BHT | n/a | 36.7% | n/a | n/a | n/a |
| Propyl Gallate | n/a | n/a | n/a | 49.6% | 68.1% |

The rate of Gonadorelin degradation was up to 2.4 times higher when no antioxidant was present, thereby justifying the addition of an antioxidant in the formulation.

1. A formulation comprising:
Gonadotrophin Releasing Hormone or an acceptable salt or analogue thereof;
progesterone or an analogue thereof; and
an acceptable carrier.

2. The formulation of claim 1, said formulation being selected from the group consisting of a solution, a micellar solution, a microemulsion, a miniemulsion or a small particle size emulsion.

3. (canceled)

4. The formulation of claim 1, said formulation being sterile.

5. The formulation of claim 4, said formulation being acceptable for injection.

6. The formulation of claim 1 wherein the progesterone or analogue is present in the formulation between about 25 mg/ml and about 150 mg/ml.

7. The formulation of claim 1 wherein the Gonadotrophin Releasing Hormone or salt or analogue thereof is present in the formulation between about 0.02 mg/ml and about 0.1 mg/ml.

8. The formulation of claim 1 additionally comprising water.

9. The formulation of claim 8 wherein the water is present between about 2 mg/ml and about 50 mg/ml.

10. The formulation of claim 1 wherein the carrier is a solvent for the progesterone or analogue thereof, or for the Gonadotrophin Releasing Hormone or salt or analogue thereof, or for both.

11. (canceled)

12. The formulation of claim 1 additionally comprising an antioxidant.

13. The formulation of claim 1 wherein the formulation has a pH of between about 4 and about 5.

14. The formulation of claim 1 wherein the the formulation additionally comprises phosphoric acid.

15. (canceled)

16. A unit dose, said unit dose comprising between about 1 ml and about 20 ml of a formulation comprising:
Gonadotrophin Releasing Hormone or an acceptable salt or analogue thereof;
progesterone or an analogue thereof; and
an acceptable carrier.

17. The unit dose of claim 16 comprising between about 2 ml and about 5 ml of the formulation.

18. The unit dose of claim 16, said unit dose being sterile.

19. The unit dose of claim 16, said unit dose being housed within a container, said container having a sufficient volume for the required quantity of the formulation.

20. (canceled)

21. A process for making a formulation comprising combining:
a solution comprising Gonadotrophin Releasing Hormone or an acceptable salt or analogue thereof dissolved in water,
a solvent, and
progesterone or an analogue thereof.

22-34. (canceled)

35. A method for increasing the probability that a female animal becomes pregnant, comprising administering to said animal a unit dose of a formulation on day 1, said formulation comprising:
Gonadotrophin Releasing Hormone or an acceptable salt or analogue thereof;
progesterone or an analogue thereof; and
an acceptable carrier.

36. The method of claim 35 additionally comprising administering to the animal prostaglandin, or an analogue thereof having similar biological activity, on day 7.

37. The method of claim 36 additionally comprising administering Gonadotrophin Releasing Hormone or an analogue or salt thereof on day 9.

38. The method of claim 35 wherein artificial insemination is conducted on day 9 or 10.

39. The method of claim 35 wherein the administering occurs during or following a hormone intervention program.

40. The formulation of claim 1 wherein the Gonadotrophin Releasing Hormone or an acceptable salt or analogue thereof is selected from the group consisting of: buserelin, deslorelin, avorelin, leuprolide, natural luteinizing hormone releasing hormone, triptorelin, nafarelin, goselerin, fertirelin or salts thereof.

41. The unit dose of claim 16 comprising about 5 ml of the formulation.
